(19) United States
(12) Patent Application Publication
(10) Pub. No.: US 2013/0084245 A1
(43) Pub. Date: Apr. 4, 2013

(54) PET MONITORING OF A-BETA-DIRECTED IMMUNOTHERAPY

(71) Applicants: Ronald Black, Audubon, PA (US); Michael Grundman, San Diego, CA (US)

(72) Inventors: Ronald Black, Audubon, PA (US); Michael Grundman, San Diego, CA (US)

(73) Assignees: Wyeth LLC, Madison, NJ (US); Janssen Alzheimer Immunotherapy, County Cork (IE)

(21) Appl. No.: 13/580,866

(22) PCT Filed: Feb. 25, 2011
(86) PCT No.: PCT/US11/26365

§ 371 Date: Nov. 5, 2012

Related U.S. Application Data
(60) Provisional application No. 61/308,253, filed on Feb. 25, 2010.

(51) Int. Cl. A61K 51/04

(52) U.S. Cl. CPC A61K 51/04
USPC 424/1.89; 424/1.81

(57) ABSTRACT

The present invention provides methods of monitoring Aβ-directed immunotherapy. The methods involve administering a PET ligand that binds to amyloid deposits and detecting the PET ligand in the brain to provide an indication of the level and/or distribution of amyloid deposits. Surprisingly, the data in the present application show that a statistically significant reduction in amyloid deposits occurs early and consistently among patients following initiation of treatment before statistically significant effects of most if not all other markers are detectable. In consequence, the present methods allow early detection of whether a patient is responding to the Aβ-directed immunotherapy and if necessary adjustment of the immunotherapy regime.

Alzheimer's disease (AD) is a progressive disease resulting in senile dementia. See generally Selkoe, TINS 16:403 (1993); Hardy et al., WO 92/13069; Selkoe, J. Neuropathol. Exp. Neurol. 53:438 (1994); Duff et al., Nature 373:476 (1995); Games et al., Nature 373:523 (1995). Broadly speaking, the disease falls into two categories: late onset, which occurs in old age (65+ years) and early onset, which develops well before the senile period, i.e., between 35 and 60 years. In both types of disease, the pathology is the same but the abnormalities tend to be more severe and widespread in cases beginning at an earlier age. The disease is characterized by senile plaques, neurofibrillary tangles and cerebral neuronal loss. Neurofibrillary tangles are intracellular deposits of microtubule associated tau protein consisting of two filaments twisted about each other in pairs. Senile plaques (i.e., amyloid plaques) are areas of disorganized neuropile up to 150 μm across with extracellular amyloid deposits at the center which are visible by microscopic analysis of sections of brain tissue. The accumulation of amyloid plaques within the brain is also associated with Down's syndrome and other cognitive disorders.

The principal constituent of the plaques is a peptide termed Aβ or β-amyloid peptide. Aβ peptide is a 4-kDa internal fragment of 39-43 amino acids of a larger transmembrane glycoprotein named amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form.

Both active and passive immunotherapy regimes against Aβ have been reported to reduce cerebral Aβ deposits in transgenic mice (Schroeter et al., J. Neurosci. 28:6787-6793 (2008); Bard et al., Nat. Med. 6:916-919 (2000)), block the synaptotoxic effects of Aβ oligomers (Shankar, et al., Nat. Med. 14:837-842 (2008)) and inhibit cognitive decline (Morgan et al., Nature. 2000; 408: 982). Analysis of cerebral amyloid deposits following immunotherapy in mice has been performed after sacrifice of the mice. A human patient receiving immunotherapy in a clinical trial has also been subject to a post-mortem analysis showing a low residual level of amyloid deposits (Nicoll, Nat. Med. 9:448-452 (2003)). Post-mortem analysis of deposits following treatment does not allow monitoring and adjustment of treatment in a patient. Other indicia of treatment measurable from body fluids, MRI or cognitive functions can be measured in living patients but may change only slowly so that meaningful differences become evident only after lengthy periods of treatment and/or when a comparison is performed across large populations of treated and control patients. PET imaging with small molecule ligands, particularly PiB has been reported to detect elevated amyloid deposits in subjects with Alzheimer's diseases. Rabinovici, Behav. Neurol. 9:117-28 (2009). However, the extent and consistency with which these deposits change in living human patients in response to immunotherapy has not been reported.

### BRIEF SUMMARY OF THE INVENTION

The invention provide methods of monitoring treatment of a patient receiving Aβ-directed immunotherapy. The methods involves administering to the patient a small-molecule positron-emission-tomography ligand (PET ligand) that binds to an amyloid deposit comprising Aβ and detecting the PET ligand in the brain using PET to provide an indication of a level of amyloid deposits of Aβ in the brain of the patient.

In some methods, the PET ligand binds to the Congo-Red binding site of Aβ. In some metho the PET ligand binds to the 2-(1-{6-[(2-fluoroethyl-(methyl)amino]-2-naphthyl}ethylidene)malononitrile (FDDNP) binding site of Aβ.

In some methods, the PET ligand is selected from the group consisting of:

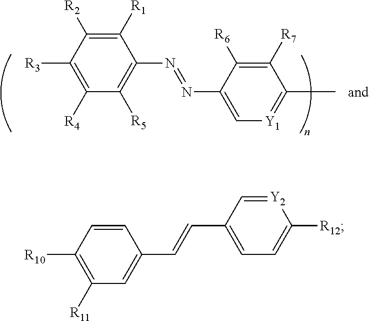

or a pharmaceutically acceptable salt thereof, wherein:

each R¹ is independently selected from the group consisting of H, NH₂ and OH;

R² is CO₂H;

R³ is OH; or R² and R³ are combined with the atoms to which they are attached to form a fused benzene ring, optionally substituted with from 1-3 substituents selected from the group consisting of SO₃H and NH₂;

each R⁴ and R⁵ are independently selected from the group consisting of H and SO₃H; or R⁴ and R⁵ are combined with the atoms to which they are attached to form a fused benzene ring;

each R⁶ and R⁷ are independently selected from the group consisting of H or CH₃;

Y′ is selected from the group consisting of CR⁸, N and N-M, wherein M is a metal selected from the group consisting of Zn, Ni, Cu and Cd; and R⁸ is H or has the formula:

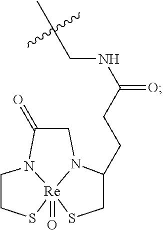

Y² is selected form the group consisting of CR⁹ and N;

R⁹ is selected from the group consisting of H, OMe, Br and I;

R¹⁰ is selected from the group consisting of −OR^{10a} and —NHCH₃;

R^{10a} is selected from the group consisting of H and Me;

each R¹¹ is selected from the group consisting of H, OH, OMe and CO₂H;

each R¹² is selected from the group having a formula:

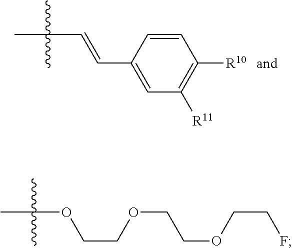

the subscript n is an integer of 1 or 2; and if n is 1, the bond extending through the right parentheses indicates a bond to H;

the wavy line indicates the point of attachment to the rest of the molecule; and

at least one atom of the PET ligand is replaced or substituted with a radiolabel selected from the group consisting of ¹¹C, ¹³N, ¹⁵O, ¹⁸F or ¹²³I.

In some methods, the PET ligand is selected from the group consisting of:

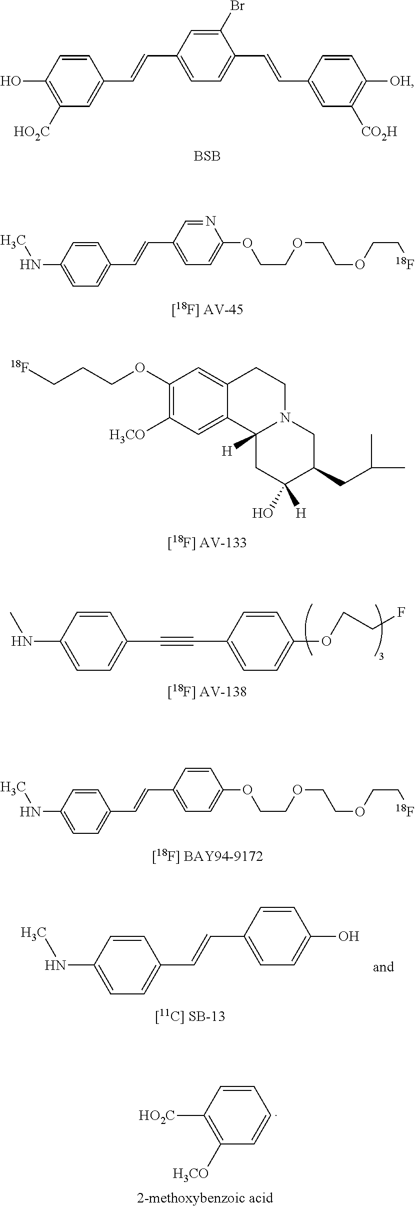

In some methods the PET ligand has a formula selected from the group consisting of:

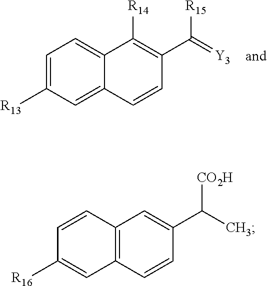

or a pharmaceutically acceptable salt thereof, wherein

R¹³ is selected from the group consisting of —N(CH₃)₂ and —N(CH₃)CH₂CH₂F;

R¹⁴ is —H;

R¹⁵ is selected from the group consisting of —CH₃ and —C(CH₃)₃; or R¹⁴ and R¹⁵ are combined with the atoms to which they are attached to form a fused cyclohexyl ring;

Y³ is selected form the group consisting of O or C(CN)₂;

R¹⁶ is selected from the group consisting of —OCH₃ and —CH₂CH(CH₃)₂; and

at least one atom of the PET ligand is replaced or substituted with a radiolabel selected from the group consisting of ¹¹C, ¹³N, ¹⁵O, ¹⁸F or ¹²³I.

In some methods, the PET ligand has a formula selected from the group consisting of:

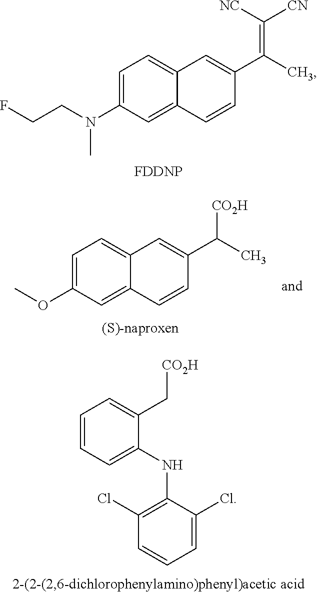

In some methods, the PET ligand has a formula selected from the group consisting of:

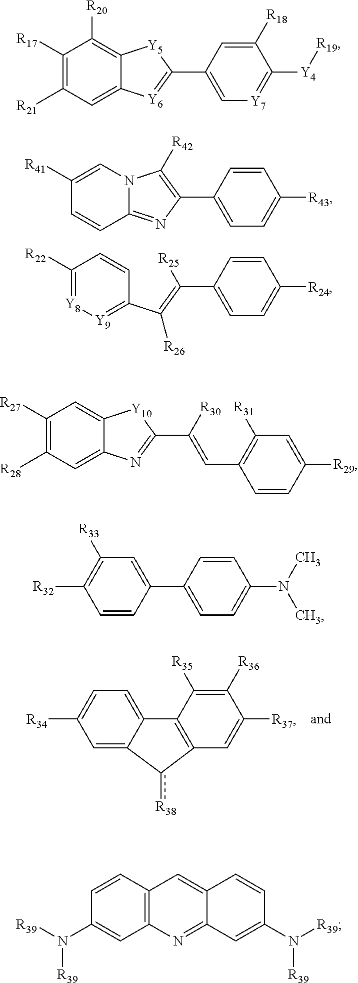

or a pharmaceutically acceptable salt thereof, wherein:

R¹⁷ is selected from the group consisting of H, CH₃, OH, OCH₃, O(CH₂)₂F, OCH₂OCH₃, CO₂CH₃, CN, NH₂, Br, I and NO₂;

Y⁴ is O or NR^{4a}; wherein R^{4a} is selected from the group consisting of H and CH₃;

Y⁵ is selected from the group consisting of S and O;

Y⁶ is selected from the group consisting of CH, N and NCH₃;

Y⁷ is selected from the group consisting of N, CH and CF;

R¹⁸ is selected from the group consisting of H, F and I;

R¹⁹ is selected from the group consisting of H, CH₃, (CH₂)ₘF and CH₂(C₆H₄)F; or when Y₄ is NR^{4a}, R^{4a} and R¹⁹ are combined with the nitrogen to which they are attached to form a morpholinyl or 4-methylpiperidinyl ring;

the subscript m is an integer of 2, 3, or 4;

R²⁰ is selected from the group consisting of H and I;

R²¹ is selected from the group consisting of Br and I;

R²² is selected from the group consisting of H, F, Br, I, CO₂CH₃ and —OR^{22a};

R^{22a} is selected from the group consisting of H, CH₃,

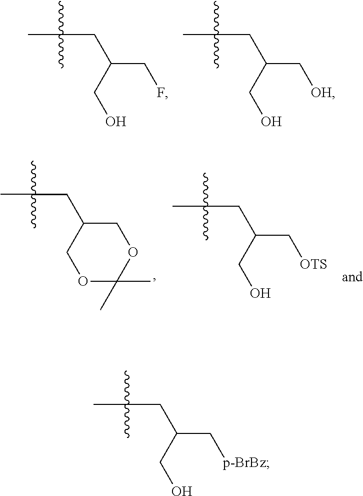

Y⁸ is selected from the group consisting of N and CR²³;

R²³ is selected from the group consisting of H and I;

R²⁴ is selected from the group consisting of H, OH, OCH₃, SCH₃, SO₂CH₃ and —N(R^{24a})(R^{24b});

each of R^{24a} and R^{24b} is independently selected from the group consisting of H and CH₃;

Y⁹ is CH or N;

each of R²⁵ and R²⁶ is independently selected from the group consisting of H and CH₃;

R²⁷ is selected from the group consisting of H, CH₃, OH, O(CH₂)₂F and F;

R²⁸ is selected from the group consisting of H, F and I;

Y¹⁰ is S or O;

R²⁹ is selected from the group consisting of F, Cl and —N(R^{29a})(R^{29b});

each of R^{29a} and R^{29b} is independently selected from the group consisting of H, CH₃ and CH₂CH₃;

each R³⁰ and R³¹ is H or are combined with the atoms to which they are attached to form a fused benzene ring;

R³² is selected from the group consisting of CH₃, Br, I, OH, NO₂, NH₂, NHCH₃ and N(CH₃)₂;

R³³ is selected from the group consisting of H and I;

R³⁴ is selected from the group consisting of H, Br, I, NH₂ and N(CH₃)₂;

each R³⁵, R³⁶ and R³⁷ is independently selected from the group consisting of H and N(R^{37a})(R^{37b});

each of R^{37a} and R^{37b} is independently selected from the group consisting of H and CH₃;

R³⁸ is selected from the group consisting of OH and O; wherein the dashed bond indicates the presence of a single bond when R³⁸ is OH and a double bond when R³⁸ is O;

R³⁹ is selected from the group consisting of CH₃ and CH₂CH₃;

R⁴⁰ is —CH₂CH₂F;

R⁴¹ is selected from the group consisting of I, Br, CH₃ and H;

R⁴² is selected from the group consisting of H and I;

R⁴³ is selected from the group consisting of −OR^{43a}, —NR^{43a}R^{43b} and —Br;

R^{43a} is selected from the group consisting of H, CH₃, CH₂CH₂F, CH₂CH₂F, or when R⁴³ is —NR^{43a}R^{43b} are combined with the nitrogen to which each is attached to form a morpholinyl group; and

at least one atom of the PET ligand is replaced or substituted with a radiolabel selected from the group consisting of ¹¹C, ¹³N, ¹⁵O, ¹⁸F or ¹²³I.

In some methods, the PET ligand is selected from the group consisting of:

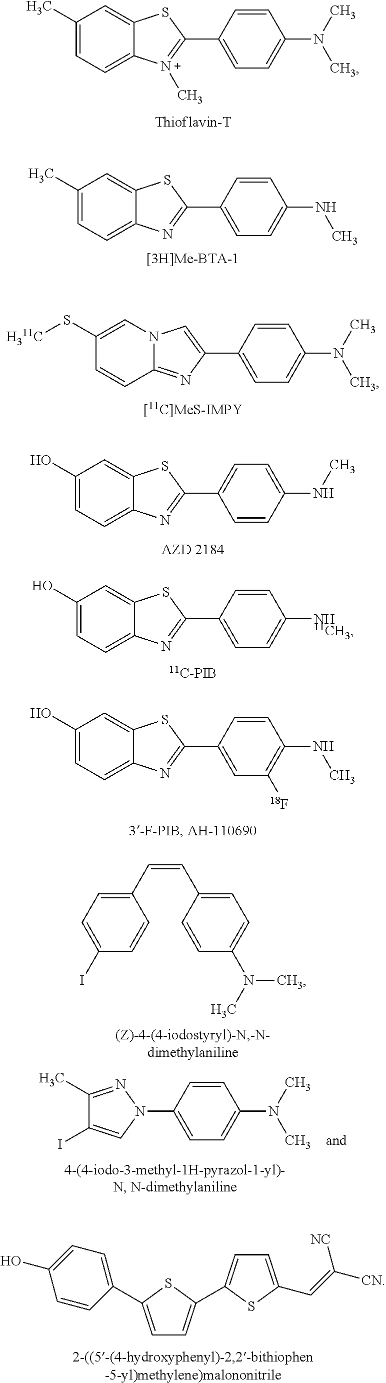

In some methods, the PET ligand is selected from the group consisting of [¹⁸F]AV-14, [¹⁸F]AV-144, [¹¹C]AZD2995, [¹⁸F]-AZD4694 and [¹⁸F]SMIBR-W372.

In some methods, the indication is a multidimensional image of levels of amyloid deposits of Aβ in the brain of the patient. In some methods, the PET ligand preferentially binds amyloid deposits relative to soluble Aβ. In some methods, the PET ligand binds fibrillar amyloid. In some methods, the PET ligand is C¹¹-PiB. In some methods, the PET ligand is administered peripherally, e.g., intravenously.

In some methods, the PET ligand is administered at a dose of 12-18 mCi. In some methods, the administering and detecting steps are before and after commencement of the Aβ-directed immunotherapy, and the level of amyloid deposits of Aβ is reduced after commencement of the therapy.

In some methods, no significant change in a biomarker selected from the group consisting of FDG, BBSI, VBSI, CSF Aβ42, CSF tau and CSF p-tau is detectable when the reduced level of amyloid deposits of Aβ is detected. In some methods, no significant increase in a measure of cognitive function is detectable when the reduced level of amyloid deposits of Aβ is detected. In some methods, the administering and detecting steps are performed with a frequency of quarterly to every two years after commencement of the Aβ-directed immunotherapy.

In some methods, the administering and detecting steps are performed on at least a first and a second occasions, the first occasion is before commencing Aβ-directed immunotherapy and the second occasion is between 9-18 months thereafter, and wherein the level of amyloid deposits of Aβ of is reduced between the first and second occasions. In some methods, the administering and detecting steps are performed before commencing Aβ-directed immunotherapy and 78 weeks thereafter.

Some methods also involve performing an MRI or CAT scan and superimposing the image of amyloid deposits of Aβ on an MRI or CAT image of the brain of the patient.

In some methods, the regime of Aβ-directed immunotherapy is adjusted in response to the monitoring. In some methods, the immunotherapy is adjusted without regard to measured values, if any, of biomarkers selected from the group consisting of FFDG, BBSI, VBSI, CSF Aβ42, CSF tau and CSFp-tau and measured values, if any, of cognitive function. In some methods, no significant effect of the Aβ-directed immunotherapy on a biomarker selected from the group consisting of FFDG, BBSI, VBSI, CSF Aβ42, CSF tau and CSFp-tau is detectable when the regime is adjusted. In some methods, no significant effect of the Aβ-directed immunotherapy on a measure of cognitive functioning is detectable when the regime is adjusted. In some methods, the regime of Aβ-directed immunotherapy is adjusted from an induction regime that reduces the detected levels of amyloid deposits of Aβ to a maintenance regime that maintains the reduced levels of amyloid deposits of Aβ responsive to the monitoring. In some methods, the dose of bapineuzumab is reduced from 1 mg/kg to 0.5 mg/kg. In some methods, the monitoring provides an indication that the levels of amyloid deposits of Aβ in the brain have increased over a period of at least 18 months following commencement of the Aβ-directed immunotherapy, and the Aβ-directed immunotherapy is terminated in response to the monitoring. In some methods, the monitoring provides an indication that the Aβ-directed immunotherapy has in a period of 18 months following commencement of therapy resulted in a positive but suboptimal response in reducing or inhibiting further increases in the levels of amyloid deposits of Aβ and the dose or frequency of administration is increased in response to the monitoring.

In some methods, the Aβ-directed immunotherapy is effected by administering bapineuzumab and the dose of bapineuzumab is increased from 0.1-0.5 mg/kg to 1 mg/kg in response to the monitoring. In some methods, the frequency of bapineuzumab administration is increased in response to the monitoring. In some methods, the detected level of amyloid deposits of Aβ in the brain increases or remains unchanged after commencing therapy, and the patient is thereafter administered an increased dose or frequency of bapineuzumab. In some methods, the Aβ-directed immunotherapy is effected by administering bapineuzumab and the level of amyloid deposits of Aβ in the brain is detected to be reduced by at least 10% relative to baseline after commencing therapy.

In some methods, the Aβ-directed immunotherapy is effected by administration of an antibody that binds to an N-terminal epitope of Aβ. In some methods, the Aβ-directed immunotherapy is effected by administration of bapineuzumab to the patient. In some methods, the Aβ-directed immunotherapy is effected by administration of AAB-003 to the patient. In some methods, the Aβ-directed immunotherapy is effected by administration of an Aβ fragment linked to a heterologous carrier as a conjugate to the patient. In some methods, the Aβ fragment is Aβ1-7 and the carrier is optionally CRM197. In some methods, the Aβ fragment is Aβ16-23, and the carrier is optionally CRM197.

In some methods, the Aβ-directed immunotherapy is selected from the group consisting of the catalytic antibody ABP 102 (Abzyme, from Abiogen Pharma); ACI-01 Ab7 C2 (AC Immune Genentech); AZD-3102 (AstraZeneca/Dyax); IVIg (Gammagard S/D Immune Globulin Intravenous (Human), from Baxter Bioscience); BAN 2401 (BioArctic Neuroscience AB/Eisai Co. Ltd.; R1450 (Hoffman-La Roche/MorphoSys); LY2062430 (Eli Lilly); h3D6 (Eli Lilly); ACU-5A5 (α ADDL mAb from Merck/Acumen); α-amyloidspheroid (ASPD) antibody (Mitsubishi Pharma Corp.); the antibody derived from PBMCs of an AN1792 patient (Neurimmune Therapeutics AG); BC05 (Takeda); the CEN701-CEN706 antibodies (Centocor/Johnson & Johnson); and PF-04360365 (also called RN-1219 (h2286), from Pfizer/Rinat Neurosciences).

In some methods, the Aβ-directed immunotherapy is effected by administration of an antibody that binds to aggregated Aβ to the patient. In some methods, the Aβ-directed immunotherapy is effected by administration of an antibody that binds to soluble Aβ to the patient.

In some methods, the PET ligand is detected in the anterior cingulate, posterior cingulate, frontal, temporal, parietal and/or occipital cortice of the brain. In some methods, the image represents levels of amyloid deposits of Aβ on a color scale.

In some methods, the patient is an ApoE4 carrier. In some methods, the patient is a non-ApoE4 carrier.

Some methods also involve administering 2-[fluorine-18]fluoro-2-deoxy-D-glucose (FDG) to the patient and detecting the FDG in the brain using PET to provide an image of brain metabolism of glucose.

The invention further provides methods of performing a clinical trial. Such methods involve assigning a population of no more than 50 patients having or at elevated risk of a disease characterized by amyloid deposits comprising Aβ in the brain to treatment and placebo groups; administering Aβ-directed therapy to the treatment group and a placebo to the placebo group; and comparing amyloid deposits in the treatment and placebo groups before and after administration of treatment or placebo by PET scanning of a small molecule PET ligand that binds amyloid deposits comprising Abeta; wherein the amyloid deposits in the treatment group are significantly reduced relative to the amyloid deposits in the placebo group. Optionally, the population consists of 10-30 patients.

The invention further provides methods of prophylaxis against Alzheimer's disease, comprising: determining a level of amyloid deposits in the brain of a patient who has no known cognitive impairment or has mild cognitive impairment but has not been diagnosed with Alzheimer's disease by PET scanning of a small molecule PET ligand that binds amyloid deposits comprising Aβ; and administering Aβ-directed immunotherapy to the patient in response to determining that the level of amyloid deposits in the brain of the patient exceeds a normal level. In some methods, the determining is performed a plurality of times before the level of amyloid deposits in the brain of the patient is determined to exceed a normal level. In some methods, the determining is performed at intervals of 6 months to 5 years. In some methods, the determining is performed annually. In some methods, the normal level is a level is a level in the brain of the patient measured in a region of the brain not associated with development of deposits in Alzheimer's disease. In some methods, the normal level is a level in a population of patients not having or at elevated risk of Alzheimer's disease. In some methods, the determining is first performed at an age between 45 and 75 years, optionally at age 50 years. In some methods the level of amyloid deposits in the patient is reduced in response to administration of the Aβ-directed immunotherapy. In some methods, the patient remains free of Alzheimer's disease for at least ten years after administering Aβ-directed immunotherapy.

### DEFINITIONS

The basic antibody structural unit comprises a tetramer of subunits. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 more amino acids. (See generally, Fundamental Immunology (Paul, W., ed., 2nd ed. Raven Press, N.Y., 1989), Ch. 7 (incorporated by reference in its entirety for all purposes).

The variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989).

References to an antibody or immunoglobulin include intact antibodies and binding fragments thereof. Typically, fragments compete with the intact antibody from which they were derived for specific binding to an antigen. Fragments include separate heavy and light chains, Fab, Fab′ F(ab′)2, Fabc, and Fv. Separate chains include NANOBODIES™ (i.e., the isolated VH fragment of the heavy chain of antibodies from camels or llamas, optionally humanized). Isolated VH fragments can also be obtained from other sources, such as human antibodies. Fragments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibody. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. (See, e.g., Songsivilai & Lachmann, Clin. Exp. Immunol. 79:315-321 (1990); Kostelny et al., J. Immunol. 148, 1547-1553 (1992).)

Unless otherwise apparent from the context, PET ligands and antibodies for use in immunotherapy bind specifically to a target. Specific binding refers to the binding of a compound to a target (e.g., as component of a plaque) that is detectably higher in magnitude and distinguishable from non-specific binding occurring to at least one unrelated target, such as for example, other targets within the brain unrelated to amyloid deposits, and particularly targets associated with unrelated diseases. Specific binding can be the result of formation of bonds between particular functional groups or particular spatial fit (e.g., lock and key type) whereas nonspecific binding is usually the result of van der Waals forces. Specific binding does not however imply that a compound binds one and only one target. Thus, a compound can and often does show specific binding of different strengths to several different targets and only nonspecific binding to other targets. Preferably, different degrees of specific binding (as for example to Aβ in different states of aggregation) can be distinguished from one another as can specific binding from nonspecific binding. Specific binding of PET ligands or antibodies to Aβ usually involves an association constant of 10⁶, 10⁷, 10⁸ or 10⁹ M⁻¹ or higher.

The term “humanized antibody” refers to an antibody that includes at least one humanized antibody chain (i.e., at least one humanized light or heavy chain and usually both). The term “humanized antibody chain” refers to an antibody chain (i.e., a light or heavy chain, respectively) having a variable region that includes a variable region framework substantially from a human antibody sequence (mature, germline or a consensus sequence) and complementarity determining regions (CDRs) (e.g., at least one CDR, preferably two CDRs, more preferably three CDRs) substantially from a non-human antibody (e.g., rodent, and optionally, mouse), and further includes constant regions entirely or substantially from a human antibody constant region. CDRs are typically as defined by Kabat, but alternatively can be as defined by Chothia or a composite of the CDR regions defined by Kabat and Chothia.

The phrase “substantially from a human antibody sequence” or “substantially human” means that, when aligned to a human antibody amino sequence (mature, germline or consensus) for comparison purposes, the region shares at least 80-90% (e.g., at least 90%), preferably 90-95%, more preferably 95-99% identity (i.e., local sequence identity) with the human framework or constant region sequence, with departures from 100% identity being the result, for example of conservative substitutions, consensus sequence substitutions, germline substitutions, backmutations, and the like. The introduction of conservative substitutions, consensus sequence substitutions, germline substitutions, backmutations, and the like, is often referred to as “optimization” of a humanized antibody or chain. The phrase “substantially from a non-human antibody” or “substantially non-human” means having an immunoglobulin or antibody sequence at least 80-95%, preferably 90-95%, more preferably, 96%, 97%, 98%, or 99% identical to that of a non-human organism, e.g., a mouse.

The term “chimeric antibody” refers to an antibody whose light and heavy chain variable regions derive from a first species (e.g., mouse or rat) and whose constant regions derive from a second species (usually human).

The term “epitope” refers to a site on an antigen to which an immunoglobulin or antibody (or antigen binding fragment thereof) specifically binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, G. E. Morris, Ed. (1996).

Antibodies that recognize the same epitope can be identified in a simple immunoassay showing the ability of one antibody to block the binding of another antibody to a target antigen, i.e., a competitive binding assay. Competitive binding is determined in an assay in which the immunoglobulin under test inhibits specific binding of a reference antibody to a common antigen, such as Aβ. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA (Moldenhauer et al., Scand. J. Immunol. 32:77 (1990). Typically, such an assay involves the use of purified antigen bound to a solid surface or cells bearing either of these, an unlabelled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Multiple isoforms of APP exist, for example APP⁶⁹⁵, APP⁷⁵¹ and APP⁷⁷⁰. Unless otherwise apparent from the context, amino acids within APP are assigned numbers according to the sequence of the APP⁷⁷⁰ isoform (see e.g., GenBank Accession No. P05067). The sequences of Aβ peptides and their relationship to the APP precursor are illustrated by FIG. 1 of Hardy et al., TINS 20, 155-158 (1997). For example, Aβ42 has the sequence:

(SEQ ID NO: 1)
H₂N-Asp-Ala-Glu-Phe-Arg-His-Asp-Ser-Gly-Tyr-Glu-
Val-His-His-Gln-Lys-Leu-Val-Phe-Phe-Ala-Glu-Asp-
Val-Gly-Ser-Asn-Lys-Gly-Ala-Ile-Ile-Gly-Leu-Met-
Val-Gly-Gly-Val-Val-Ile-Ala-OH.

An N-terminal epitope of Aβ means an epitope with residues 1-11. An epitope within a C-terminal region means an epitope within residues 29-43, and an epitope within a central regions means an epitope with residues 12-28.

Monomeric Aβ and small oligomeric assemblies of about 4-10 monomers, sometimes known as ADDLs (Lambert et al., PNAS May 26, 1998 vol. 95 no. 11 6448-6453), are soluble in aqueous solution, including body fluids, such as CSF. Higher order assemblies of Aβ formed by in vitro aggregation or in vivo in the form of plaques are substantially insoluble in aqueous solutions. Aggregated Aβ is believed to be held together at least in part, by hydrophobic residues at the C-terminus of the peptide (part of the transmembrane domain of APP). Higher order insoluble deposits are sometimes referred to as amyloid fibrils. Fibrils are characterized by a cross-beta structure and are substantially insoluble even in detergents and denaturing solvents (see Schmidt et al., PNAS 106, 19813-19818 (2009): Cai et al., anent Medicinal Chemistry 24, 19-52 (2007)).

The term “Fc region” refers to a C-terminal region of an IgG heavy chain. Although the boundaries of the Fc region of an IgG heavy chain can vary slightly, an Fc region is typically defined as spanning from about amino acid residue Cys226 to the carboxyl-terminus of an IgG heavy chain(s).

The term “effector function” refers to an activity that resides in the Fc region of an antibody (e.g., an IgG antibody) and includes, for example, the ability of the antibody to bind effector molecules such as complement and/or Fc receptors, which can control several immune functions of the antibody such as effector cell activity, lysis, complement-mediated activity, antibody clearance, and antibody half-life. Effector function can also be influenced by mutations in the hinge region.

The term “Kabat numbering” is defined as the numbering of the residues as in Kabat et al. (Sequences of Proteins of Immunological Interest, 5th Ed. Public Health Service, National Institutes of Health, Bethesda, Md. (1991)), incorporated herein by reference.

The term “adjuvant” refers to a compound that when administered in conjunction with an antigen elicits and/or augments an immune response to the antigen, but when administered alone does not generate an immune response to the antigen. Adjuvants can augment an immune response by several mechanisms including lymphocyte recruitment, stimulation of B and/or T cells, and stimulation of macrophages.

The term “ApoE4 carrier” is sometimes used to refer to patients having one or two ApoE4 alleles and “ApoE4 noncarrier”, ApoE4 non-carrier” or “non-ApoE4 carrier” to refer to patients having zero ApoE4 alleles.

Mild Cognitive Impairment can be diagnosed by the 2001 guidelines of the American Academy of Neurology. In brief, these guidelines require an individual's report of his or her own memory problems, preferably confirmed by another person; measurable, greater-than-normal memory impairment detected with standard memory assessment tests; normal general thinking and reasoning skills and ability to perform normal daily activities.

An individual at elevated risk of Alzheimer's disease or other disease characterized by amyloid deposits of Aβ in the brain is one having one or more known risk factors (e.g., >70 years old, genetic, biochemical, family history, prodromal symptoms) placing the subject at significantly higher risk than the general population of developing the disease in a defined period, such as five years.

Statistical significance refers to p≦0.05. A change in marker relative to a baseline measurement of the marker is considered significant if the change is outside a typical margin of error in repeated measurement. For measurement of amyloid deposits by PET scanning, a typical margin of error (e.g., reproducibility of measurement on the same patient) is about 5%.

A positive treatment response means either a reduction in amyloid deposits or an inhibition of further increase in amyloid response as would occur in a patient not receiving Aβ-directed immunotherapy.

PET ligands or agents used in immunotherapy can be formulated as free acids or bases or as pharmaceutically acceptable salts (see generally Berget al., 66 J. PHARM. SCI. 1-19 (1977), and C. G. Wermuth and P. H. Stahl (eds.) “Pharmaceutical Salts: Properties, Selection, and Use” Verlag Helvetica Chimica Acta, 2002 [ISBN 3-906390-26-8]. Pharmaceutically acceptable salts substantially retain the biologic activity of the free acid or base. Pharmaceutical salts tend to be more soluble in aqueous and other protic solvents than are the corresponding free acid or base forms. Pharmaceutically acceptable acid salts include hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include aluminum, calcium, lithium, magnesium, potassium, sodium, zinc, and diethanolamine salts.

### BRIEF DESCRIPTION OF THE DRAWINGS

FIG. 1 shows the estimated mean change from baseline over time on Pittsburgh Compound-B positron emission tomography ([¹¹C]PiB PET) average through week 78. Data points represent least squares means with respective 95% confidence intervals. *Difference between placebo- and bapineuzumab-treated patients at week 78=−0.24; p=0.003.

FIG. 2 shows plots for each patient in the modified intent-to-treat population showing the Pittsburgh Compound-B positron emission tomography ([¹¹C]PiB PET) average at baseline, [¹¹C]PiB PET average at last available visit, and change in [¹¹C]PiB PET average from baseline to last available visit. Filled circles indicate patients with data at week 78; open circles indicate patients with their last available visit prior to week 78. Horizontal lines represent the means for each treatment group.

FIG. 3 shows Pittsburgh Compound-B positron emission tomography ([¹¹C]PiB PET) images in two bapineuzumab-treated (A, B) and two placebo-treated (C, D) patients. Average [¹¹C]PiB PET changes from baseline to week 78 are shown at the top center of each panel for each patient (A through D). The scale bar shows the PiB uptake ratios relative to cerebellum by color. The pre- and post-treatment scans are from magnetic resonance imaging co-registered images in the same plane.

### DETAILED DESCRIPTION OF THE INVENTION

### I. General

The present invention provides methods of monitoring Aβ-directed immunotherapy. The methods involve administering a PET ligand that binds to amyloid deposits and detecting the PET ligand in the brain to provide an indication of the level and/or distribution of amyloid deposits. Surprisingly, the present data show that a statistically significant reduction in amyloid deposits occurs early and consistently among patients following initiation of treatment before statistically significant effects of most if not all other markers are detectable. In consequence, the present methods allow early detection of whether a patient is responding to the Aβ-directed immunotherapy and if necessary adjustment of the immunotherapy regime.

### II. PET Ligands

Positron emission tomography (PET) is a noninvasive imaging technique that permits spatial and temporal imaging true quantitation of PET ligand concentrations in tissues. The technique involves the use of PET ligands (also known as radiotracers), labeled with positron or gamma emitting radionuclides.

The most commonly used positron emitting radionuclides are ¹⁵O, ¹³N, ¹¹C and ¹⁸F, which are accelerator produced and have half lives of 2, 10, 20 and 110 minutes respectively. The most widely used gamma emitting radionuclides are ¹⁸F, ⁹⁹ mTc, ²⁰¹TI and ¹²³I. Fluorine-18 has some advantages over carbon-11: (1) it has lower positron energy than carbon-11 (0.63 5 vs. 0.96 MeV); (2) because of the long half-life of fluorine-18, the PET studies can be performed for more than 2 hours if necessary; (3) the long half-life is convenient for radiosynthesis; and (4) the radioligands can be transported off-site when a cyclotron is not available.

PET scanning detects gamma rays emitted by a positron-emitting radionuclide (tracer), which is introduced into the body on a biologically active molecule. A PET ligand for use in the present methods includes such a radionuclide as a component of or otherwise linked to a moiety that binds to a component of amyloid deposits. Thus, PET ligands for use in the present methods bind to Aβ or other component of amyloid deposits found in Alzheimer's and related diseases characterized by deposits of Aβ in the brain. Amyloid deposits are aggregates of Aβ present in the brain of Alzheimer's patients, also known as plaques and are substantially insoluble in aqueous solution, such as body fluids (e.g., the CSF). More mature plaques are also substantially insoluble in detergents and denaturing solvents (for example, prolonged incubation in 6 M guanidine-HCl at room temperature does not result in quantitative solubilization). Amyloid deposits can also be formed by in vitro aggregation of Aβ to an insoluble mass. Amyloid deposits have a β-pleated sheet structure and stain with Congo Red and or Thioflavin-T dye. PET ligands of the invention preferably bind to Aβ in the form of amyloid deposits with an affinity of at least 10⁷, 10⁸ or 10⁹M⁻¹. The affinity of binding to a deposit may depend on whether the deposit is formed in vitro, in a transgenic animal or is obtained from a human. An affinity of at least 10⁷, 10⁸ or 10⁹ M⁻¹ for amyloid deposits isolated from humans is preferred. Binding to amyloid deposits in the brain serves to immobilize a PET ligand to an insoluble structure in the brain and allows development of an image. Some PET ligands preferentially binds to fibrillar amyloid over other forms of amyloid deposits. PET ligands may or may not bind to soluble forms of Aβ, i.e., monomer and small oligomeric assemblies, sometimes known as ADDLs, as well as binding to amyloid deposits. Some PET ligands preferentially bind to soluble forms of Aβ over aggregated forms. Such forms can be used for imaging soluble forms of Aβ that are immobilized in the brain through attachment to other structures, such as neurons. PET ligands to VMAT-2 (Swiss-Prot accession number Q05940) can also be used.

PET ligands are preferably small molecules meaning they have a molecular weight less than 1000 Da and preferably less than 500 Da. PET ligands are preferably able to cross the blood brain barrier to allow peripheral administration.

Several PET ligands have been developed for imaging of Aβ that are directed to one of three type of binding sites for Aβ: Congo-Red (CR), Thioflavin-T (Th-T) and 2-(1-{6-[(2-fluoroethyl-(methyl)amino]-2-naphthyl}ethylidene)malononitrile (FDDNP) (Cai et al. Current Medicinal Chemistry, 2007, 14, 19-52). Binding to the same or overlapping epitope on Aβ as Congo Red, Thioflavin-T or FDDNP can be recognized by a competition assay between a PET ligand and Congo Red or Thioflavin-T. Examples of CR-type PET ligands have the following general formulae:

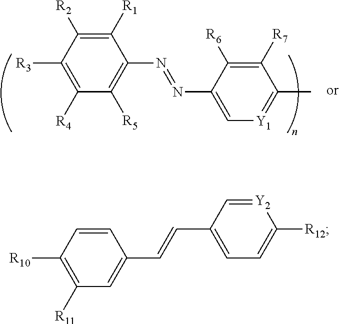

wherein:
each R¹ is independently selected from the group consisting of H, NH₂ and OH;

### R² is CO₂H;

R³ is OH; or R² and R³ are combined with the atoms to which they are attached to form a fused benzene ring, optionally substituted with from 1-3 substituents selected from the group consisting of SO₃H, and NH₂;
each R⁴ and R⁵ are independently selected from the group consisting of H and SO₃H; or R⁴ and R⁵ are combined with the atoms to which they are attached to form a fused benzene ring;
each R⁶ and R⁷ are independently selected from the group consisting of H or CH₃;
Y′ is selected from the group consisting of CR⁸, N and N-M, wherein M is a metal selected from the group consisting of Zn, Ni, Cu and Cd; and R⁸ is H or has the formula:

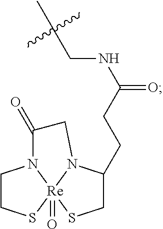

Y² is selected form the group consisting of CR⁹ and N;
R⁹ is selected from the group consisting of H, OMe, Br and I;
R¹⁰ is selected from the group consisting of —OR^{10a} and —NHCH₃;
R^{10a} is selected from the group consisting of H and Me;
each R¹¹ is selected from the group consisting of H, OH, OMe and CO₂H;
each R¹² is selected from the group having a formula:

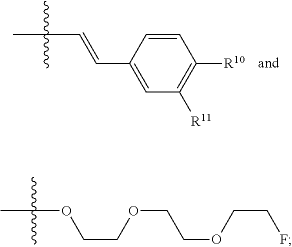

the subscript n is an integer of 1 or 2; and if n is 1, the bond extending through the right parentheses indicates a bond to H; and
the wavy line indicates the point of attachment to the rest of the molecule.

Within any of the embodiments of the present invention, at least one atom of the PET ligand is replaced or substituted with a radiolabel selected from the group consisting of ¹¹C, ¹³N, ¹⁵O, ¹⁸F or ¹²³I.

Specific examples of CR PET ligands include (trans, trans)-1-bromo-2,5-bis-(3-hydroxycarbonyl-4-hydroxy)styryl-benzene (BSB), [¹⁸F]AV-19, [¹⁸F]AV-45, [¹⁸F]AV-133, [¹⁸F]AV-138, [¹⁸F]AV-144, BAY 94-9172 and 2-methoxybenzoic acid. These compounds have the following formulae:

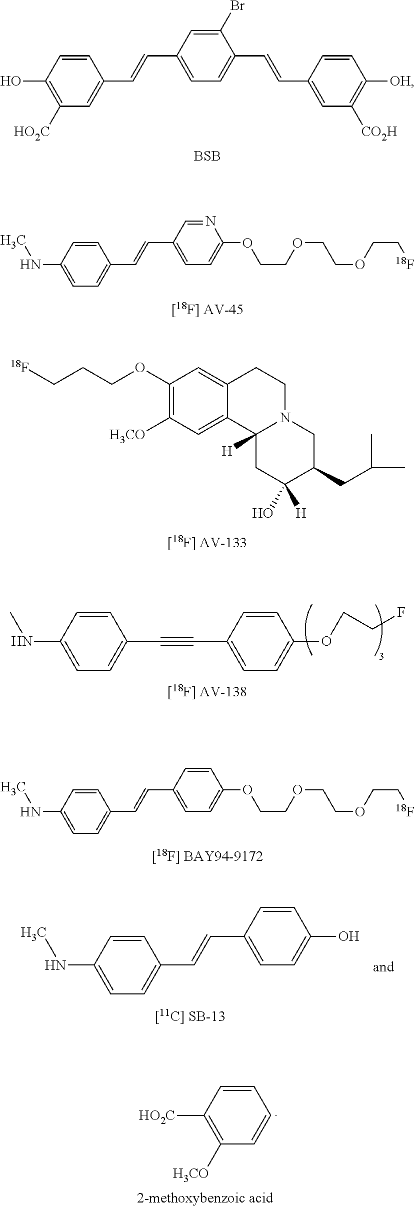

Examples of FDDNP-type PET ligands have the following general formulae:

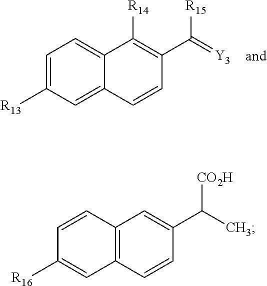

or a pharmaceutically acceptable salt thereof, wherein
R¹³ is selected from the group consisting of —N(CH₃)₂ and —N(CH₃)CH₂CH₂F;

### R¹¹ is H;

R¹⁵ is selected from the group consisting of —CH₃ and —C(CH₃)₃; or R¹¹ and R¹⁵ are combined with the atoms to which they are attached to form a fused cyclohexyl ring;
Y³ is selected form the group consisting of O or C(CN)₂;
R¹⁶ is selected from the group consisting of —OCH₃ and —CH₂CH(CH₃)₂.

Specific examples of FDDNP PET ligands include: 2-(1-{6-[(2-fluoroethyl-(methyl)amino]-2-naphthyl}ethylidene)malononitrile (FDDNP); (S)-2-(6-methoxy-2-naphthyl)propionic acid ((S)-naproxen), and 2-(2-(2,6-dichlorophenylamino)phenyl)acetic acid. These compounds have the following formulae:

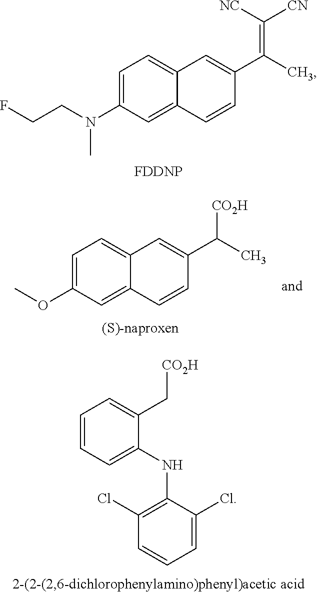

Examples of Th-T-type PET ligands have the following general formulae:

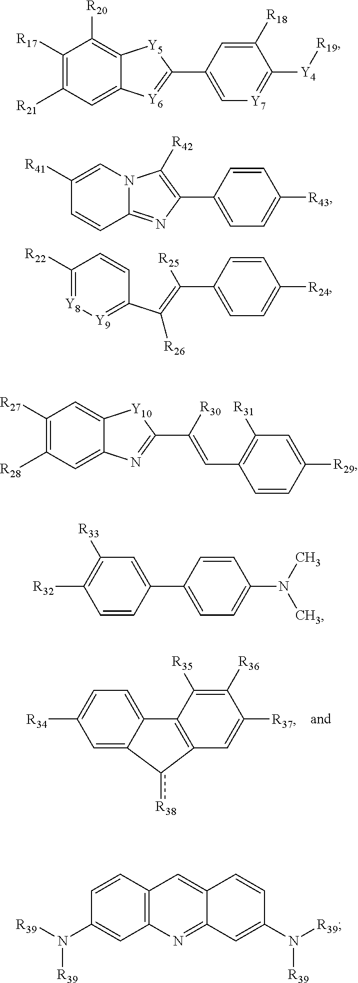

or a pharmaceutically acceptable salt thereof, wherein:
R¹⁷ is selected from the group consisting of H, CH₃, OH, OCH₃, O(CH₂)₂F, OCH₂OCH₃, CO₂CH₃, CN, NH₂, Br, I and NO₂;
Y⁴ is O or NR^{4a}; wherein R^{4a} is selected from the group consisting of H and CH₃;
Y⁵ is selected from the group consisting of S and O;
Y⁶ is selected from the group consisting of CH, N and NCH₃;
Y⁷ is selected from the group consisting of N, CH and CF;
R¹⁸ is selected from the group consisting of H, F and I;
R¹⁹ is selected from the group consisting of H, CH₃, (CH₂)ₘF and CH₂(C₆H₄)F; or when Y₄ is NR^{4a}, R^{4a} and R¹⁹ are combined with the nitrogen to which they are attached to form a morpholinyl or 4-methylpiperidinyl ring;
the subscript m is an integer of 2, 3, or 4;
R²⁰ is selected from the group consisting of H and I;
R²¹ is selected from the group consisting of Br and I;
R²² is selected from the group consisting of H, F, Br, I, CO₂CH₃ and OR^{22a};
R^{22a} is selected from the group consisting of H, CH₃,

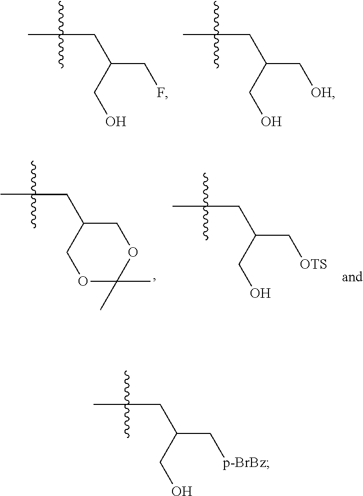

Y⁸ is selected from the group consisting of N and CR²³;
R²³ is selected from the group consisting of H and I;
R²⁴ is selected from the group consisting of H, OH, OCH₃, SCH₃, SO₂CH₃ and N(R^{24a})(R^{24b});
each of R^{24a} and R^{24b} is independently selected from the group consisting of H and CH₃;

### Y⁹ is CH or N;

each of R²⁵ and R²⁶ is independently selected from the group consisting of H and CH₃;
R²⁷ is selected from the group consisting of H, CH₃, OH, O(CH₂)₂F and F;
R²⁸ is selected from the group consisting of H, F and I;

### Y¹⁰ is S or O;

R²⁹ is selected from the group consisting of F, Cl and N(R^{29a})(R^{29b});
each of R^{29a} and R^{29b} is independently selected from the group consisting of H, CH₃ and CH₂CH₃;
each R³⁰ and R³¹ is H or are combined with the atoms to which they are attached to form a fused benzene ring;
R³² is selected from the group consisting of CH₃, Br, I, OH, NO₂, NH₂, NHCH₃ and N(CH₃)₂;
R³³ is selected from the group consisting of H and I;
R³⁴ is selected from the group consisting of H, Br, I, NH₂ and N(CH₃)₂;
each R³⁵, R³⁶ and R³⁷ is independently selected from the group consisting of H and N(R^{37a})(R^{37b});
each of R^{37a} and R^{37b} is independently selected from the group consisting of H and CH₃;
R³⁸ is selected from the group consisting of OH and O; wherein the dashed bond indicates the presence of a single bond when R³⁸ is OH and a double bond when R³⁸ is O;
R³⁹ is selected from the group consisting of CH₃ and CH₂CH₃;

### R⁴⁰ is —CH₂CH₂F;

R⁴¹ is selected from the group consisting of I, Br, CH₃ and H;
R⁴² is selected from the group consisting of H and I;
R⁴³ is selected from the group consisting of —OR^{43a}, —NR^{43a}R^{43b} and —Br;
R^{43a} is selected from the group consisting of H, CH₃, CH₂CH₂F and CH₂CH₂F, or when R⁴³ is —NR^{43a}R^{43b} are combined with the nitrogen to which each is attached to form a morpholinyl group.

Specific examples of Th-T PET ligands include: (2-[6-(methylamino)pyridin-3-yl]-1,3-benzothiazol-6-ol), (AZD2184); [S-methyl-¹¹C] N,N-Dimethyl-4-(6-(methylthio)imidazo[1,2-a]pyridine-2-yl)aniline, (¹¹C[MeS-IMPY]); 2-(4′-methylaminophenyl)-6-hydroxybenzothiazole (6-OH-BTA-1, ¹¹C-PIB); [¹⁸F]-2-(4′-methylamino-3′-fluorophenyl)-6-hydroxybenzothiazole (3′-F-PIB, AH-110690); Thioflavin T; 2-[4′-([3H]methylamino)phenyl]-6-methylbenzothiazole ([3H]Me-BTA-1); (Z)-4-(4-iodostyryl)-N,N-dimethylaniline; 4-(4-iodo-3-methyl-1H-pyrazol-1-yl)-N,N-dimethylaniline; 2#5′-(4-hydroxyphenyl)-2,2′-bithiophen-5-yl)methylene)malononitrile, [¹¹C]AZD2995, [¹⁸F]-AZD4694 (All AstraZeneca) and [¹⁸F]-SMIBR-W372 (Siemens). These compounds have the following formulae:

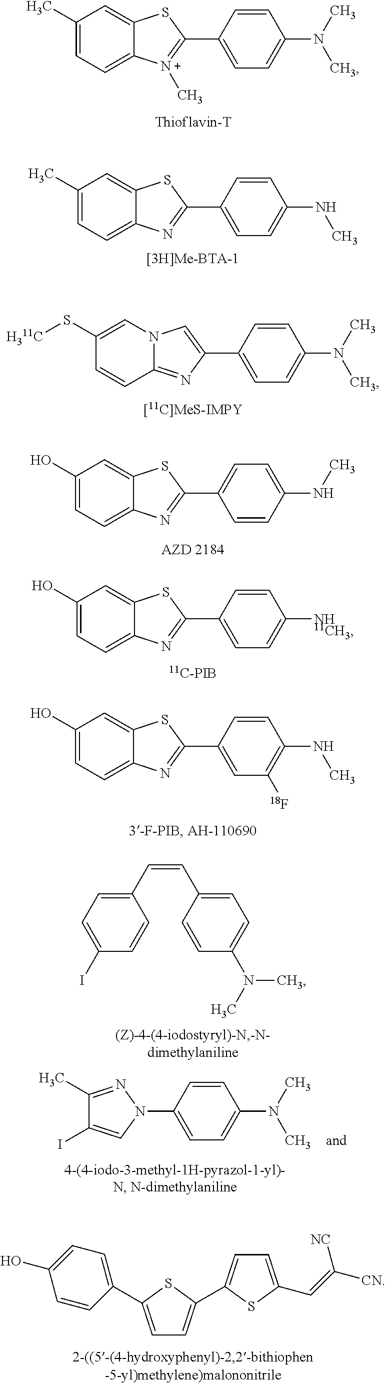

One such compound, Pittsburgh Compound-B ([¹¹C]PiB). (Klunk et al., Ann Neurol 55(3):306-319 (2004); Ikonomovic et al., Brain; 131:1630-1645 (2008)) is an exemplary PET ligand. PiB is thioflavin-analogue that binds to aggregated fibrillar deposits of the Aβ peptide with low nanomolar affinity, enters the brain in amounts sufficient for imaging with PET, and clears rapidly from normal brain tissue. (Price et al., J. Cereb. Blood Flow Metab. 25:1528-1547 (2005)). At the low nanomolar concentrations typically used in PET studies, the binding of PiB to postmortem human brain has been shown to be selective for fibrillar Aβ deposits. (Ikonomovic et al., supra; Fodero-Tavoletti et al., J Neurosci; 27:10365-10371 (2007)). Compared with controls, AD patients show approximately two-fold retention of [¹¹C]PiB in areas of brain association cortex known pathologically to be targeted by Aβ deposits. [¹¹C]PiB retention is equivalent in AD patients and controls in areas known to be relatively unaffected by Aβ deposition (such as subcortical white matter, pons, and cerebellum).

Other PET ligands that can be used include the Th-T PET ligand ¹⁸F-AH110690 (a 3′-fluoro analog of PIB from GE Healthcare, also known as flutemetamol); and two CR PET ligands: the stilbene derivative ¹⁸F-BAY94-9172 (Bayer Schering Pharma) (which performed comparably to ¹¹C-PIB in a preliminary study in AD and controls [Rowe, Lancet Neurol. 2008; 7(2):129-3535]), and (E)-4-(2-(6-(2-(2-(2-(2-18F-fluoroethoxy)ethoxy)ethoxy)pyridin-3-yl)vinyl)-N-methyl benzenamine (¹⁸F-AV-45 from Avid Radiopharmaeuticals) [Klunk, Curr Opin Neurol. 2008; 21(6):683-732, Rowe, supra, Nordberg, Neuropsychologia. 2008; 46(6):1636-41].

### III. PET Imaging

PET ligands are usually administered to a patient to the systemic circulation by a peripheral route, with intravenous administration being preferred. PET ligands can thus be delivered by the systemic circulation across the blood brain barrier to come into contact with amyloid deposits. PET ligand binding to an amyloid deposit in the brain is immobilized and can be detected in a subsequent PET scan. Unbound PET ligand or PET ligand bound to soluble Aβ is cleared from the brain more rapidly than bound PET scan and is not detected or is detected to a lesser extent relative to the same amount of bound PET ligand.

The dose of PET ligand administered can be measured by radioactivity. An exemplary dose, particularly for [¹¹C]PiB, is 12-18 μCi.

The interval between administering the PET ligand and performing the scan can depend on the PET ligand and particularly its rate of uptake and clearing into the brain, and the half-life of its radiolabel. The interval can be, for example, about 10-120 min or 30-90 min.

A PET scan can be performed using, for example, a conventional PET imager and auxiliary equipment. The scan typically includes one or more regions of the brain known in general to be associated with deposits in Alzheimer's disease and one or more regions in which few if any deposits are generally present to serve as controls. Regions of the brain associated with presence of amyloid deposits in Alzheimer's disease include, for example, the anterior cingulated, posterior cingulated, frontal, temporal, parietal or occipital cortice of the brain. Regions of the brain associated with lack of deposits include, for example, subcortical white matter, pons, and the cerebellum.

The detected signal can be represented as a multidimensional image. The multidimensional image can be in two dimensions representing a cross-section through the brain, in three dimensions, representing the three dimensional brain or in four dimensions representing changes in the three dimensional brain over time. A color scale can be used with different colors indicating different amounts of label and inferentially amyloid deposit detected. The results of the scan can also be presented numerically as in Table 2 or 3 with numbers relating to the amount of label detected and consequently amount of amyloid deposits. The label present in a region of the brain known to be associated with deposits in Alzheimer's disease can be compared with the label present in a region known not to be associated with deposits to provide a ratio indicative of the extent of deposits within the former region. For the same radiolabeled ligand, such ratios provide a comparable measure of amyloid deposits and changes thereof between different patient. For example, if the ratio of label in a region of the brain known to have deposits to a region known to lack deposits is 2:1 (normalizing for any difference in volume of the regions) before commencing immunotherapy and thereafter the ratio is reduced to 1.75:1, it can be concluded that the immunotherapy has reduced amyloid deposits by 0.25 units or 25% of the difference in label (i.e., 2−1=1 unit) between the regions of the brain known to be associated and not associated with amyloid deposits in Alzheimer's disease. In a more general case, the percentage decrease in amyloid deposits can be represented as I_{B}−I_{T}/I_{B}−I_{C}×100%, where I_{B} is the baseline intensity of signal in a region of the brain associated with deposits, I_{T} is the signal after treatment in the region of the brain associated with deposits, and I_{C} is the intensity in a control region of the brain not associated with deposits.

In some methods, a PET scan is performed concurrent with or in the same patient visit as an MRI or CAT scan. An MRI or CAT scan provides more anatomical detail of the brain than a PET scan. However, the image from a PET scan can be superimposed on an MRI or CAT scan image more precisely indicating the location of PET ligand and inferentially amyloid deposits relative to anatomical structures in the brain. An MRI scan is also useful for assessing whether vasogenic edema has developed (see WO 09/017,467). Some machines can perform both PET scanning and MRI or CAT scanning without the patient changing positions between the scans facilitating superimposition of images.

IV. Monitoring Immunotherapy with PET Scanning

The methods of the invention can be practiced on patients who have been diagnosed with Alzheimer's disease (e.g., clinical evaluation, patient history, and/or MRI and/or by the criteria of the Diagnostic and Statistical Manual IV). The methods can also be performed on patients with other diseases characterized by amyloid deposits including Aβ in the brain or a patient at risk of such a disease. Such diseases include Alzheimer's disease, Down's syndrome, mild cognitive impairment, cerebral amyloid angiopathy (CAA), dementia with Lewy Bodies (DLB) and posterior cortical atrophy (PCA).

To limit the exposure of a patient to radiation present on a PET ligand, scans are preferably performed at times most likely to reveal information useful in maintaining or adjusting the immunotherapy regime before the patient's condition has deteriorated when further treatment has little if any benefit. Typically a baseline measurement is performed before commencing immunotherapy. One or more subsequent scans are then performed after commencing treatment. The first such scan after commencing treatment can be performed about 3-24 months after commencing treatment. Usually, such a scan is performed within 6-18 or 9-18 months of commencing treatments, such as for example, at about 6, 9, 12, 15 or 18 months. In some methods, a scan is performed 78 weeks after treatment. Any subsequent scans (i.e., 3^{rd} and subsequent scans) can be performed at intervals of, for example, quarterly, six-monthly, yearly or every two years. In some patients no more than six scans are performed in total to limit exposure to radiation.

PET scans can also be performed as a prophylactic measure in asymptomatic patients or in patients who have symptoms of mild cognitive impairment but have not yet been diagnosed with Alzheimer's disease but are at elevated risk of developing Alzheimer's disease. For asymptomatic patients, scans are particularly useful for individuals considered at elevated risk of Alzheimer's disease because of a family history, genetic (e.g., ApoE4, APP717 or APPSwe) or biochemical risk factors (e.g., elevated CSF t-tau or p-tau or reduced CSF Aβ42) or mature age. Prophylactic scans can commence for example, at a patient age between 45 and 75 years. In some patients, a first scan is performed at age 50 years. Above normal levels of amyloid may start to develop up to 20 years before onset of even mild cognitive impairment. Prophylactic scans can be performed at intervals of for example, between six months and ten years, preferably between 1-5 years. In some patients, prophylactic scans are performed annually If a PET scan performed as a prophylactic measure indicates abnormally high levels of amyloid deposits, immunotherapy can be commenced and subsequent PET scans performed as in patients diagnosed with Alzheimer's disease. If a PET scanned performed as a prophylactic measure indicates levels of amyloid deposits within normal levels, further PET scans can performed at intervals of between six months and 10 years, and preferably 1-5 years, as before, or in response to appearance of signs and symptoms in Alzheimer's disease or mild cognitive impairment. By combining prophylactic scans with administration of Aβ-directed immunotherapy if and when an above normal level of amyloid deposits is detected, levels of amyloid deposits can be reduced to at or closer to normal levels or at least inhibited from increasing further, and the patient can remain free of Alzheimer's disease for a longer period than if not receiving prophylactic scans and Aβ-directed immunotherapy (e.g., at least 5, 10, 15 or 20 years, or for the rest of the patient's life).

Normal levels of amyloid deposits are levels of amyloid deposits in the brains of a representative sample of individuals in the general population who have not been diagnosed with Alzheimer's disease (or other disease characterized by amyloid deposits of Aβ in the brain) and are not considered at elevated risk of developing such disease (e.g., a representative sample of disease-free individuals under 50 years of age). Alternatively, a normal level can be recognized in an individual patient if the PET signal according to the present methods in a region of the brain in which amyloid deposits are known to develop is not different (within the accuracy of measurement) from the signal from a region of the brain in which it is known that such deposits do not normally develop. An elevated level in an individual can be recognized by comparison to the normal levels (e.g., outside mean and variance of a standard deviation) or simply from an elevated signal beyond experimental error in a region of the brain associated with amyloid deposits compared with a regions not known to be associated with deposits. For purposes of comparing the levels of amyloid deposits in an individual and population, the amyloid deposits should preferably be determined in the same region(s) of the brain, these regions including at least one region in which amyloid deposits associated with Alzheimer's or related disease are known to form. A patient having an elevated level of amyloid deposits is a candidate for commencing immunotherapy.

After commencing immunotherapy, effects of immunotherapy on amyloid deposits can be first seen in the period of about 3-24 months, and more typically 6-18 months. The effect is most readily observed as a decrease in amyloid deposits. The observed decrease over an 18 month period can be for example in the range of 1-100, 1-50 1-25, 5-100, 5-50, 5-25, 5-15, 10-100, 10-50, 10-25 or 10-15, 15-100, 15-50 or 15-25%% of the baseline value, for a example, 5-15% reduction. Such effects can be measured in one or more regions of the brain in which deposits are known to form or can be measured from an average of such regions (see, e.g., Table 3) Such a decrease can almost always be attributed as a treatment effect because amyloid deposits do not usually decrease in the absence of treatment. The total effect of treatment can be approximated by adding to the percentage reduction relative to baseline the increase in amyloid deposits that would occur in an average untreated patient (e.g., about 15%). Thus, for example, a 5-35% reduction relative to baseline value in a treated patient, corresponds to a reduction of 20-50% of the amyloid deposits that would form in a typical treated patient.

Maintenance of amyloid deposits at an approximately constant level or even a small increase in amyloid deposits can also be an indication of response to treatment albeit a suboptimal response. Such responses can be compared with a time course of levels of amyloid deposits in patients with Alzheimer's disease not receiving treatment to determine whether the immunotherapy is having an effect in inhibiting further increases of amyloid deposits. In the present examples, individuals not receiving treatment showed an average increase of about 15% in amyloid deposits over 18 months. Thus, even an increase in amyloid deposits approaching 15% after 18 months of treatment may be consistent with a positive but suboptimal response to treatment.

In at least some patients, effects of immunotherapy on amyloid deposits by way of a decrease in amyloid deposits in one or more regions of the brain associated with amyloid deposits, or deposits remaining at a constant level or increasing more slowly than in untreated patients are discernable before significant changes in other signs or symptoms of Alzheimer's disease. Such other signs or symptoms that may be preceded by detectable effects on amyloid deposits measured by PET include various cognitive measures (ADAS-CO11, ADAS-0012, DAASD, CDR-SB, NTB, NPI, MMSE), [18F]FDG, MRI markers (BBSI and VBSI), and CSF markers AFx-42, tau and phosphor-tau. Thus, PET imaging can be used to monitor treatment with or without contemporaneous measurements of any of these markers.

After performing the scan, it has been found that the radioactivity concentrates in the urine of the patient. The patient is therefore preferably instructed to empty his or her bladder within two hours of the scan.

### V. Adjustments in Immunotherapy Regime Based on Monitoring

Monitoring of changes in amyloid deposits allows adjustment of the immunotherapy regime in response to the treatment. PET monitoring provides an indication of the nature and extent of response to treatment. Then a determination can be made whether to adjust treatment and if desired treatment can be adjusted in response to the PET monitoring. As indicated above, such as indication is usually evident in a period from about 3-24 months, or 6-18 or 9-18 months after commencing treatment. Because amyloid levels change more rapidly than all or most other markers, treatment can be adjusted based on amyloid level without measurement of other parameters of treatment noted above, or with measurement but without obtaining any evidence of significant change in such parameters, or without reliance on other markers. PET monitoring thus allows for Aβ-directed immunotherapy to be adjusted before other biomarkers, MRI or cognitive measures have detectably responded (e.g., assessed as in the present examples). A significant change means that comparison of the value of a parameter after treatment relative to basement provides some evidence that treatment has or has not resulted in a beneficial effect. In some instances, a change of values of a parameter in a patient itself provides evidence that treatment has or has not resulted in a beneficial effect. In other instances, the change of values, if any, in a patient, is compared with the change of values, if any, in a representative control population of patients not undergoing immunotherapy. A difference in response in a particular patient from the normal response in the control patient (e.g., mean plus variance of a standard deviation) can also provide evidence that an immunotherapy regime is or is not achieving a beneficial effect in a patient.

In some patients, monitoring indicates a detectable decline in amyloid level but that amyloid level remains above normal. For example, in the present examples, amyloid levels declined by about 10% over 18 months. In such patients, if there are no unacceptable side effects, the treatment regime can be continued as is or even increased in frequency of administration and/or dose if not already at the maximum recommended dose.

If the monitoring indicates an amyloid level in a patient has already been reduced to at or near a normal level of amyloid, the immunotherapy regime can be adjusted from one of induction (i.e., that reduces the level of amyloid deposits) to one of maintenance (i.e., that maintains amyloid at an approximately constant level). Such a regime can be effected by reducing the dose and or frequency of administering immunotherapy. For example, if an induction regime involves administering an antibody such as bapineuzumab at a dosage of 1 mg/kg by quarterly intravenous administration, a maintenance regime could involve administering a reduced dose of 0.1-0.5 mg/kg antibody quarterly.

In other patients, monitoring can indicate that immunotherapy is having some beneficial effect but a suboptimal effect. An optimal effect can be defined as a percentage reduction in amyloid level within the top half or quartile of the change in amyloid deposits (measured or calculated over the whole brain or representative region(s) thereof in which amyloid deposits are known to form) experienced by a representative sample of patients undergoing immunotherapy at a given time point after commencing therapy. A patient experiencing a smaller decline or a patient whose amyloid level remains constant or even increases but to a lesser extent than expected in the absence of immunotherapy (e.g., as inferred from a control group of patients not administered immunotherapy) can be classified as experiencing a positive but suboptimal response. Such patients can optionally be subject to an adjustment of regime in which the dose and or frequency of administration of an agent is increased. For example, in the case of administration of an antibody, such as bapineuzumab, the dose can be increased from 0.1-0.5 mg/kg to 1 mg/kg.

In some patients, amyloid deposits may increase in similar or greater fashion to amyloid deposits in patients not receiving immunotherapy. If such increases persist over a period of time, such as 18 months or 2 years, even after any increase in the frequency or dose of agents, immunotherapy can if desired be discontinued in favor of other treatments.

### VI. Clinical Trials

Use of PET scanning to detect amyloid deposits provides an end point by which the efficacy of immunotherapy regimes can be assessed in a clinical trial. Such assessment can be expected to reach statistical significance from relatively small patient populations e.g., no more than 15, 25, 50 or 100 patients (split between treated and placebo groups). Some clinical trials enroll 15-30 patients. Statistical significance can also be seen relatively soon after commencing immunotherapy for example at a period of no more than 6, 9, 12, 15, 18, 21 or 24 months.

### VII. Aβ-Directed Immunotherapy

Aβ-directed immunotherapy means the administration of an antibody that specifically binds to Aβ or an agent that induces such an antibody, such an a fragment of Aβ. Various agents and regimes are described in e.g., WO2009/052439 incorporated by reference, and summarized below.

### A. Passive Immunotherapy

A variety of antibodies to Aβ have been described in the patent and scientific literature for use in immunotherapy of Alzheimer's disease, some of which are in clinical trials (see, e.g., U.S. Pat. No. 6,750,324). Such antibodies can specifically bind to an N-terminal epitope, a mid (i.e., central)-epitope or a C-terminal epitope as defined above. Some antibodies are N-terminal specific (i.e., such antibodies specifically bind to the N-terminus of Aβ without binding to APP). As noted above antibodies binding to epitopes within residues 1-10, 1-3, 1-4, 1-5, 1-6, 1-7 or 3-7 of Aβ42 or within residues 2-4, 5, 6, 7 or 8 of Aβ, or within residues 3-5, 6, 7, 8 or 9 of Aβ, or within residues 4-7, 8, 9 or 10 of Aβ42 can be used. Some antibodies are C-terminal specific (i.e., specifically bind to a C-terminus of Aβ without binding to APP). Antibodies can be polyclonal or monoclonal. Polyclonal sera typically contain mixed populations of antibodies specifically binding to several epitopes along the length of APP. However, polyclonal sera can be specific to a particular segment of Aβ such as Aβ1-11) without specifically binding to other segments of Aβ. Preferred antibodies are chimeric, humanized (including veneered antibodies) (see Queen et al., Proc. Natl. Acad. Sci. USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539), or human (Lonberg et al., WO 93/12227 (1993); U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741 (1991)) EP1481008, Bleck, Bioprocessing Journal 1 (September/October 2005), US 2004132066, US 2005008625, WO 04/072266, WO 05/065348, WO 05/069970, and WO 06/055778.

3D6 antibody, 10D5 and variants thereof are examples of antibodies that can be used. Both are described in US 20030165496, US 20040087777, WO 02/46237, and WO 04/080419, WO 02/088306 and WO 02/08830 and U.S. Pat. No. 7,318,9237. 10D5 antibodies are also described in US 20050142131. Additional 3D6 antibodies are described in US 20060198851 and PCT/US05/45614. 3D6 is a monoclonal antibody (mAb) that specifically binds to an N-terminal epitope located in the human β-amyloid peptide, specifically, residues 1-5. 10D5 is a mAb that specifically binds to an N-terminal epitope located in the human β-amyloid peptide, specifically, residues 3-6. A cell line producing the 3D6 monoclonal antibody (RB96 3D6.32.2.4) was deposited with the American Type Culture Collection (ATCC), Manassas, Va. 20108, USA on Apr. 8, 2003 under the terms of the Budapest Treaty and assigned accession number PTA-5130. A cell line producing the 10D5 monoclonal antibody (RB44 10D5.19.21) was deposited with the ATCC on Apr. 8, 2003 under the terms of the Budapest Treaty and assigned accession number PTA-5129.

Bapineuzumab (International Non-Proprietary Name designated by the World Health Organization) means a humanized 3D6 antibody comprising a light chain having a mature variable region having the amino acid sequence designated SEQ ID NO: 2 and a heavy chain having a mature variable region having the amino acid sequence designated SEQ ID NO: 3. (The heavy and light chain constant regions of the antibody designated bapineuzumab by WHO are human IgG1 and human kappa respectively.)

Humanized 3D6 Light Chain Variable Region
(SEQ ID NO: 2)
Asp Val Val Met Thr Gln Ser Pro Leu Ser Leu Pro
Val Thr Pro Gly Glu Pro Ala Ser Ile Ser Cys Lys
Ser Ser Gln Ser Leu Leu Asp Ser Asp Gly Lys Thr
Tyr Leu Asn Trp Leu Leu Gln Lys Pro Gly Gln Ser
Pro Gln Arg Leu Ile Tyr Leu Val Ser Lys Leu Asp
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Trp Gln Gly
Thr His Phe Pro Arg Thr Phe Gly Gln Gly Thr Lys
Val Glu Ile Lys
Humanized 3D6 Heavy Chain Variable Region
(SEQ ID NO: 3)
Glu Val Gln Leu Leu Glu Ser Gly Gly Gly Leu Val
Gln Pro Gly Gly Ser Leu Arg Leu Ser Cys Ala Ala
Ser Gly Phe Thr Phe Ser Asn Tyr Gly Met Ser Trp
Val Arg Gln Ala Pro Gly Lys Gly Leu Glu Trp Val
Ala Ser Ile Arg Ser Gly Gly Gly Arg Thr Tyr Tyr
Ser Asp Asn Val Lys Gly Arg Phe Thr Ile Ser Arg
Asp Asn Ser Lys Asn Thr Leu Tyr Leu Gln Met Asn
Ser Leu Arg Ala Glu Asp Thr Ala Val Tyr Tyr Cys
Val Arg Tyr Asp His Tyr Ser Gly Ser Ser Asp Tyr
Trp Gly Gln Gly Thr Leu Val Thr Val Ser Ser

Another exemplary antibody is 12A11 or a chimeric or humanized or nanobody form thereof. The 12A11 antibody or a variant thereof, is described in US 20050118651, US 20060198851, WO 04/108895, and WO 06/066089, all of which are incorporated by reference in their entirety herein for all purposes. 12A11 is a mAb that specifically binds to an N-terminal epitope located in the human β-amyloid peptide, specifically, residues 3-7. A cell line producing the 12A11 monoclonal antibody was deposited at the ATCC (American Type Culture Collection, 10801 University Boulevard, Manassas, Va. 20110-2209) on Dec. 12, 2005 and assigned ATCC accession number PTA-7271.

Sequences for the light and heavy chain variable regions (not including signal sequences) of an exemplary humanized 12A11 antibody are as follows:

Light chain
(SEQ ID NO: 4)
Asp Val Val Met Thr Gln Ser Pro Leu Ser Leu Pro
Val Thr Pro Gly Glu Pro Ala Ser Ile Ser Cys Arg
Ser Ser Gln Ser Ile Val His Ser Asn Gly Asn Thr
Tyr Leu Glu Trp Tyr Leu Gln Lys Pro Gly Gln Ser
Pro Gln Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Phe Gln Ser
Ser His Val Pro Leu Thr Phe Gly Gln Gly Thr Lys
Leu Glu Ile Lys
Heavy chain
(SEQ ID NO: 5)
Gln Val Gln Leu Val Glu Ser Gly Gly Gly Val Val
Gln Pro Gly Arg Ser Leu Arg Leu Ser Cys Ala Phe
Ser Gly Phe Ser Leu Ser Thr Ser Gly Met Ser Val
Gly Trp Ile Arg Gln Ala Pro Gly Lys Gly Leu Glu
Trp Leu Ala His Ile Trp Trp Asp Asp Asp Lys Tyr
Tyr Asn Pro Ser Leu Lys Ser Arg Leu Thr Ile Ser
Lys Asp Thr Ser Lys Asn Thr Val Tyr Leu Gln Met
Asn Ser Leu Arg Ala Glu Asp Thr Ala Val Tyr Tyr
Cys Ala Arg Arg Thr Thr Thr Ala Asp Tyr Phe Ala
Tyr Trp Gly Gln Gly Thr Thr Val Thr Val Ser Ser

Other exemplary antibodies include 12B4 antibody or variant thereof (e.g., chimeric and humanized), as described in US 20040082762A1 and WO 03/077858. 12B4 is a mAb that specifically binds to an N-terminal epitope located in the human β-amyloid peptide, specifically, residues 3-7.

Other exemplary antibodies are 6C6 antibody, or a variant thereof, e.g., chimeric and humanized, as described in a US 20060165682 and WO 06/06604. 6C6 is a mAb that specifically binds to an N-terminal epitope located in the human β-amyloid peptide, specifically, residues 3-7. A cell line producing the antibody 6C6 was deposited on Nov. 1, 2005, with the ATCC under the terms of the Budapest Treaty and assigned accession number PTA-7200.

Other exemplary antibodies are 2H3 antibody and variants thereof, e.g., chimeric or humanized, as described in US 20060257396. 2H3 is a mAb that specifically binds to an N-terminal epitope located in the human β-amyloid peptide, specifically, residues 2-7. A cell line producing the antibody 2H3 was deposited on Dec. 13, 2005, with the ATCC under the terms of the Budapest Treaty and assigned accession number PTA-7267.

Other exemplary antibodies include 3A3 and variants thereof, e.g., chimeric or humanized, as described in US 20060257396. 3A3 is a mAb that specifically binds to an N-terminal epitope located in the human β-amyloid peptide, specifically, residues 3-7. A cell line producing the antibody 3A3 was deposited on Dec. 13, 2005, with the ATCC under the terms of the Budapest Treaty and assigned accession number PTA-7269.

Other exemplary antibodies are 2B1, 1C2 or 9G8, and chimeric and humanized forms thereof. Cell lines producing the antibodies 2B1, 1C2 and 9G8 were deposited on Nov. 1, 2005, with the ATCC under the terms of the Budapest Treaty and were assigned accession numbers PTA-7202, PTA-7199 and PTA-7201, respectively.

Another exemplary antibody is a humanized 266 antibody or chimeric or humanized forms thereof. The 266 antibody binds to an epitope between residues 13-28 of Aβ. A cell line producing the antibody 266 antibody was deposited on Jul. 20, 2004 with the ATCC under the terms of the Budapest Treaty and assigned accession number PTA-6123. Humanized forms of the 266 antibody are described in US 20040265308, US 20040241164, WO 03/016467, and U.S. Pat. No. 7,195,761.

Light and heavy chain variable regions sequences of exemplary humanized 266 antibodies are shown below (not including signal sequences)

Light chain
(SEQ ID NO: 6):
Asp Xaa Val Met Thr Gln Xaa Pro Leu Ser Leu Pro
Val Xaa Xaa Gly Gln Pro Ala Ser Ile Ser Cys Arg
Ser Ser Gln Ser Leu Xaa Tyr Ser Asp Gly Asn Ala
Tyr Leu His Trp Phe Leu Gln Lys Pro Gly Gln Ser
Pro Xaa Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Xaa Gly Val Tyr Tyr Cys Ser Gln Ser
Thr His Val Pro Trp Thr Phe Gly Xaa Gly Thr Xaa
Xaa Glu Ile Lys Arg

wherein: Xaa at position 2 is Val or Ile; Xaa at position 7 is Ser or Thr; Xaa at position 14 is Thr or Ser; Xaa at position 15 is Leu or Pro; Xaa at position 30 is Ile or Val; Xaa at position 50 is Arg, Gln, or Lys; Xaa at position 88 is Val or Leu; Xaa at position 105 is Gln or Gly; Xaa at position 108 is Lys or Arg; and Xaa at position 109 is Val or Leu; and

Heavy chain
(SEQ ID NO: 7)
Xaa Val Gln Leu Val Glu Xaa Gly Gly Gly Leu Val
Gln Pro Gly Gly Ser Leu Arg Leu Ser Cys Ala Ala
Ser Gly Phe Thr Phe Ser Arg Tyr Ser Met Ser Trp
Val Arg Gln Ala Pro Gly Lys Gly Leu Xaa Leu Val
Ala Gln Ile Asn Ser Val Gly Asn Ser Thr Tyr Tyr
Pro Asp Xaa Val Lys Gly Arg Phe Thr Ile Ser Arg
Asp Asn Xaa Xaa Asn Thr Leu Tyr Leu Gln Met Asn
Ser Leu Arg Ala Xaa Asp Thr Ala Val Tyr Tyr Cys
Ala Ser Gly Asp Tyr Trp Gly Gln Gly Thr Xaa Val
Thr Val Ser Ser

wherein: Xaa at position 1 is Glu or Gln; Xaa at position 7 is Ser or Leu; Xaa at position 46 is Glu, Val, Asp, or Ser; Xaa at position 63 is Thr or Ser; Xaa at position 75 is Ala, Ser, Val or Thr; Xaa at position 76 is Lys or Arg; Xaa at position 89 is Glu or Asp; and Xaa at position 107 is Leu or Thr.

An exemplary humanized 266 antibody comprises the following light chain and heavy chain sequences (not including signal sequences).

(SEQ ID NO: 8)
Asp Val Val Met Thr Gln Ser Pro Leu Ser Leu Pro Val Thr Leu Gly Gln Pro
Ala Ser Ile Ser Cys Arg Ser Ser Gln Ser Leu Ile Tyr Ser Asp Gly Asn Ala Tyr
Leu His Trp Phe Leu Gln Lys Pro Gly Gln Ser Pro Arg Leu Leu Ile Tyr Lys Val
Ser Asn Arg Phe Ser Gly Val Pro Asp Arg Phe Ser Gly Ser Gly Ser Gly Thr
Asp Phe Thr Leu Lys Ile Ser Arg Val Glu Ala Glu Asp Val Gly Val Tyr Tyr
Cys Ser Gln Ser Thr His Val Pro Trp Thr Phe Gly Gln Gly Thr Lys Val Glu Ile
Lys Arg Thr Val Ala Ala Pro Ser Val Phe Ile Phe Pro Pro Ser Asp Glu Gln Leu
Lys Ser Gly Thr Ala Ser Val Val Cys Leu Leu Asn Asn Phe Tyr Pro Arg Glu
Ala Lys Val Gln Trp Lys Val Asp Asn Ala Leu Gln Ser Gly Asn Ser Gln Glu
Ser Val Thr Glu Gln Asp Ser Lys Asp Ser Thr Tyr Ser Leu Ser Ser Thr Leu Thr
Leu Ser Lys Ala Asp Tyr Glu Lys His Lys Val Tyr Ala Cys Glu Val Thr His
Gln Gly Leu Ser Ser Pro Val Thr Lys Ser Phe Asn Arg Gly Glu Cys
(SEQ ID NO: 9)
Glu Val Gln Leu Val Glu Ser Gly Gly Gly Leu Val Gln Pro Gly Gly Ser Leu
Arg Leu Ser Cys Ala Ala Ser Gly Phe Thr Phe Ser Arg Tyr Ser Met Ser Trp Val
Ary Gln Ala Pro Gly Lys Gly Leu Glu Leu Val Ala Gln Ile Asn Ser Val Gly
Asn Ser Thr Tyr Tyr Pro Asp Thr Val Lys Gly Arg Phe Thr Ile Ser Arg Asp
Asn Ala Lys Asn Thr Leu Tyr Leu Gln Met Asn Ser Leu Arg Ala Glu Asp Thr
Ala Val Tyr Tyr Cys Ala Ser Gly Asp Tyr Trp Gly Gln Gly Thr Leu Val Thr
Val Ser Ser Ala Ser Thr Lys Gly Pro Ser Val Phe Pro Leu Ala Pro Ser Ser Lys
Ser Thr Ser Gly Gly Thr Ala Ala Leu Gly Cys Leu Val Lys Asp Tyr Phe Pro
Glu Pro Val Thr Val Ser Trp Asn Ser Gly Ala Leu Thr Ser Gly Val His Thr Phe
Pro Ala Val Leu Gln Ser Ser Gly Leu Tyr Ser Leu Ser Ser Va Val Thr Val Pro
Ser Ser Ser Leu Gly Thr Gln Thr Tyr Ile Cys Asn Val Asn His Lys Pro Ser Asn
Thr Lys Val Asp Lys Lys Val Glu Pro Lys Ser Cys Asp Lys Thr His Thr Cys
Pro Pro Cys Pro Ala Pro Glu Leu Leu Gly Gly Pro Ser Val Phe Leu Phe Pro Pro
Lys Pro Lys Asp Thr Leu Met Ile Ser Arg Thr Pro Glu Val Thr Cys Val Val Val
Asp Val Ser His Glu Asp Pro Glu Val Lys Phe Asn Trp Tyr Val Asp Gly Val
Glu Val His Asn Ala Lys Thr Lys Pro Arg Glu Glu Gln Tyr Asn Ser Thr Tyr
Arg Val Val Ser Val Leu Thr Val Leu His Gln Asp Trp Leu Asn Gly Lys Glu
Tyr Lys Cys Lys Val Ser Asn Lys Ala Leu Pro Ala Pro Ile Glu Lys Thr Ile Ser
Lys Ala Lys Gly Gln Pro Ary Glu Pro Gln Val Tyr Thr Leu Pro Pro Ser Arg
Asp Glu Leu Thr Lys Asn Gln Val Ser Leu Thr Cys Leu Val Lys Gly Phe Tyr
Pro Ser Asp Ile Ala Val Glu Trp Glu Ser Asn Gly Gln Pro Glu Asn Asn Tyr Lys
Thr Thr Pro Pro Val Leu Asp Ser Asp Gly Ser Phe Phe Leu Tyr Ser Lys Leu
Thr Val Asp Lys Ser Arg Trp Gln Gln Gly Asn Val Phe Ser Cys Ser Val Met
His Glu Ala Leu His Asn His Tyr Thr Gln Lys Ser Leu Ser Leu Ser Pro Gly Lys

The antibody can also be 15C11 or a chimeric or humanized form thereof (see US 20060165682), which specifically binds to an epitope within Aβ15-24.

The antibody can also be a humanized form of 20C2 or a chimeric or humanized form thereof. Such antibodies are described, e.g., in US 2007081998. The core linear epitope for 20C2 corresponds to amino acid residues 3-8 of Aβ1-42, with a conformational epitope that is dependent upon elements from within residues 17-42 of Aβ.

Another antibody that can be used according to the invention is C705 or a chimeric or humanized form thereof, which binds an epitope comprising amino acids 7-12 of the Aβ peptide, as described in WO 05/028511.

Another antibody that can be used according to the invention is C706 or a chimeric or humanized form thereof, which binds to an epitope comprising amino acids 6-11 of the Aβ peptide, as described in WO 05/028511.

Other antibodies that can be used according to the invention include 2286 antibody and humanized or chimeric forms thereof. These antibodies recognize an epitope comprising amino acids 28-40 of the Aβ peptide, as described in US 20070160616.

Another exemplary antibody is 2E7 and chimeric or humanized forms thereof, as disclosed in WO 07/113,172. The 2E7 antibody binds residues 1-12 of Aβ peptide, but not 2-13, or longer variants of the peptide.

An additional antibody that can be used according to the invention includes humanized or chimeric 9TL antibody (ATCC accession numbers PTA-6124 and PTA-6125), as described in WO 06/036291.

Humanized versions of the 6G antibody can also be used according to the invention. The heavy and light chain variable regions, without signal sequences, are shown as SEQ ID NOs:104 and 11, respectively.

(SEQ ID NO: 104)
QVQLVQSGAEVKKPGASVKVSCKASGYTFTTYAIHWVRQAPGQGLEW
MGFTSPYSGVSNYNQKFKGRVTMTRDTSTSTVYMELSSLRSEDTAVYY
CARFDNYDRGYVRDYWGQGTLV
(SEQ ID NO: 115)
DIVMTQSPDSLAVSLGERATINCRASESVDNDRISFLNWYQQKPGQPPK
LLIYAATKQGTGVPDRFSGSGSGTDFTLTISSLQAEDVAVYYCQQSKEF
PWSFGGGTKVEIKRTV

Additional antibodies that can be used according to the invention are humanized versions of the 2.1 antibody, as described in WO 06/081171. These antibodies include CDRs of the murine 2.1 antibody and substitute residues from the human VKII A19/JK4 light chain variable framework region.

Other antibodies that can be used according to the invention include CW1181 and CW1185 antibodies. These antibodies specifically bind to two regions of the Aβ peptide, as described in WO 03/070760 and US 20050196399. The first region comprises AEFRHDSGY (SEQ ID NO: 12) or a fragment thereof (e.g., AEFRHD (SEQ ID NO: 13), or EFRHDSG (SEQ ID NO: 14), EFRHD (SEQ ID NO: 15)) and second region comprises the amino acid sequence YEVHHQKLVFFAEDVG (SEQ ID NO: 16) or a fragment thereof (e.g., VFFA (SEQ ID NO: 17), or QKLFFAEDV (SEQ ID NO: 18)).

An additional antibody that can be used according to the invention is the monoclonal NAB61 antibody or a chimeric or humanized form thereof. NAB61 binds Aβ1-11, but does not bind to full length APP or C99, as disclosed in WO 07/062,088. Similarly, the monoclonal 82E1 antibody can be used according to the invention. 82E1 binds the N-terminus of the Aβ peptide, but not full length APP, as disclosed in US 20080025988.

Other antibodies of the invention are anti-ADDL antibodies. Such antibodies have been generated and selected for the ability to bind ADDLs specifically, without binding to Aβ monomer or amyloid fibrils. See e.g., WO 04/031400.

Other antibodies that can be used include (i) the catalytic antibody ABP 102 (Abzyme, from Abiogen Pharma); (ii) ACI-01 Ab7 C2 (AC Immune Genentech); (iii) AZD-3102 (AstraZeneca/Dyax); (iv) IVIg (Gammagard S/D Immune Globulin Intravenous (Human), from Baxter Bioscience); (v) BAN 2401 (BioArctic Neuroscience AB/Eisai Co. Ltd.; (vi) R1450 (Hoffman-La Roche/MorphoSys); (vii) LY2062430 (Eli Lilly); (viii) h3D6 (Eli Lilly); (ix) ACU-5A5 (β-ADDL mAb from Merck/Acumen); β-amyloidspheroid (ASPD) antibody (Mitsubishi Pharma Corp.); (xi) the antibody derived from PBMCs of an AN1792 patient (Neurimmune Therapeutics AG); (xii) BC05 (Takeda); (xiii) the CEN701-CEN706 antibodies (Centocor/Johnson & Johnson); and (xiv) PF-04360365 (also called RN-1219 (h2286), from Pfizer/Rinat Neurosciences). Each of these antibodies can be used according to any of the methods of the invention.

The ABP 102 antibody cleaves aggregated Aβ as described, e.g., in U.S. Pat. No. 6,387,674 and WO 99/06536. The ACI-01 Ab7 C2 antibody binds the Aβ peptide between residues 10-20 and is described in US 20070166311. The IVIg Gammagard SD Immune Globulin antibody is described, e.g., on the Baxter Bioscience website at Baxter.com. The BAN 2401 antibody is a humanized antibody that binds Aβ protofibrils, and is described, e.g., in WO 05/123775. The human R-1450 HuCAL antibody has a dual 266/3D6 epitope. The humanized LY2062430 antibody (IgG) binds the Aβ peptide between residues 16-23, and is described, e.g., in U.S. Pat. No. 7,195,761. The humanized h3D6 antibody binds the Aβ peptide at residues 1-5, and is described, e.g., in U.S. Pat. No. 7,318,923. The BC05 antibody binds a C terminal Aβ epitope, as described by Asami-Odaka et al. (2005)Neurodegenerative Diseases 2:36-43. The CEN701-CEN706 antibodies are described, e.g., in WO 05/028511. The humanized PF-04360365 antibody binds the Aβ peptide between residues 28-40 and is described, e.g., in WO 04/032868.

Any of the antibodies or antibody fragments described herein can be designed or prepared using standard methods, as disclosed, e.g., in US 20040038304, US 20070020685, US 200601660184, US 20060134098, US 20050255552, US 20050130266, US 2004025363, US 20040038317, US 20030157579, and U.S. Pat. No. 7,335,478.

Any of the antibodies described above can be produced with different isotypes or mutant isotypes to control the extent of binding to different Fcγ receptors. Antibodies lacking an Fc region (e.g., Fab fragments) lack binding to Fcγ receptors. Selection of isotype also affects binding to Fcγ receptors. The respective affinities of various human IgG isotypes for the three Fcγ receptors, FcγRI, FcγRII, and FcγRIII, have been determined. (See Ravetch & Kinet, Annu. Rev. Immunol. 9, 457 (1991)). FcγRI is a high affinity receptor that binds to IgGs in monomeric form, and the latter two are low affinity receptors that bind IgGs only in multimeric form. In general, both IgG1 and IgG3 have significant binding activity to all three receptors, IgG4 to FcγRI, and IgG2 to only one type of FcγRII called IIa_{LR} (see Parren et al., J. Immunol. 148, 695 (1992). Therefore, human isotype IgG1 is usually selected for stronger binding to Fcγ receptors is desired, and IgG2 is usually selected for weaker binding.

Mutations on, adjacent, or close to sites in the hinge link region (e.g., replacing residues 234, 235, 236 and/or 237 with another residue) in all of the isotypes reduce affinity for Fcγ receptors, particularly FcγRI receptor (see, e.g., U.S. Pat. No. 6,624,821). Optionally, positions 234, 236 and/or 237 are substituted with alanine and position 235 with glutamine (See, e.g., U.S. Pat. No. 5,624,821.) Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235A, and G237A for human isotype IgG1. A particular preferred antibody is bapineuzumab having human isotype IgG and these three mutations of the Fc region of human IgG1. Other substitutions that decrease binding to Fcγ receptors are an E233P mutation (particularly in mouse IgG1) and D265A (particularly in mouse IgG2a). Other examples of mutations and combinations of mutations reducing Fc and/or C1q binding are described in the Examples (E318A/K320A/R322A (particularly in mouse IgG1), L235A/E318A/K320A/K322A (particularly in mouse IgG2a). Similarly, residue 241 (Ser) in human IgG4 can be replaced, e.g., with proline to disrupt Fc binding.

Additional mutations can be made to the constant region to modulate effector activity. For example, mutations can be made to the IgG2a constant region at A330S, P331S, or both. For IgG4, mutations can be made at E233P, F234V and L235A, with G236 deleted, or any combination thereof. IgG4 can also have one or both of the following mutations S228P and L235E. The use of disrupted constant region sequences to modulate effector function is further described, e.g., in WO 06/118,959 and WO 06/036291.

Additional mutations can be made to the constant region of human IgG to modulate effector activity (see, e.g., WO 06/03291). These include the following substitutions: (i) A327G, A330S, P331S; (ii) E233P, L234V, L235A, G236 deleted; (iii) E233P, L234V, L235A; (iv) E233P, L234V, L235A, G236 deleted, A327G, A330S, P331S; and (v) E233P, L234V, L235A, A327G, A330S, P331S to human IgG 1.

The affinity of an antibody for the FcR can be altered by mutating certain residues of the heavy chain constant region. For example, disruption of the glycosylation site of human IgG1 can reduce FcR binding, and thus effector function, of the antibody (see, e.g., WO 06/036291). The tripeptide sequences NXS, NXT, and NXC, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces FcR binding to the antibody.

The sequences of several exemplary humanized 3D6 antibodies and their components parts are shown below. Human constant regions show allotypic variation and isoallallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. The allotype of the IgG1 constant region shown below is 3D6 (AAB-001) is Glmz which has Glu at position 356 and Met at position 358. The allotype of the kappa constant region shown below is Km3, which has an Ala at position 153 and a Val at position 191. A different allotye Km(1) has Val and Leu at positions 153 and 191 respectively. Allotypic variants are reviewed by J Immunogen 3: 357-362 (1976) and Loghem, Monogr Allergy 19: 40-51 (1986). Other allotypic and isoallotypic variants of the illustrated constant regions are included. Also included are constant regions having any permutation of residues occupying polymorphic positions in natural allotypes. Examples of other heavy chain IgG1 allotypes include: Glm(f), Glm(a) and Glm(x). Glm(f) differs from Glm(z) in that it has an Arg instead of a Lys at position 214. Glm(a) has amino acids Arg, Asp, Glu, Leu at positions 355-358.

Humanized 3D6 Full Length Light Chain (signal sequence not included) (bapineuzumab and AAB-003)

(SEQ ID NO: 19)
DVVMTQSPLSLPVTPGEPASISCKSSQSLLDSDGKTYLNWLLQKPGQSP
QRLIYLVSKLDSGVPDRFSGSGSGTDFTLKISRVEAEDVGVYYCWQGTH
FPRTFGQGTKVEIKRTVAAPSVFIFPPSDEQLKSGTASVVCLLNNFYPR
EAKVQWKVDNALQSGNSQESVTEQDSKDSTYSLSSTLTLSKADYEKHK

Humanized 3D6 Heavy Chain, Not Including Signal Sequence (IgG1 isotype, L234A/L235A/G237A): AAB-003

(SEQ ID NO: 20)
EVQLLESGGGLVQPGGSLRLSCAASGFTFSNYGMSWVRQAPGKGLEW
VASIRSGGGRTYYSDNVKGRFTISRDNSKNTLYLQMNSLRAEDTAVYY
CVRYDHYSGSSDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPGK

The C-terminal K residue can be absent, as indicated below.

(SEQ ID NO: 21)
EVQLLESGGGLVQPGGSLRLSCAASGFTFSNYGMSWVRQAPGKGLEW
VASIRSGGGRTYYSDNVKGRFTISRDNSKNTLYLQMNSLRAEDTAVYY
CVRYDHYSGSSDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPEAAGAPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG.

Full-length heavy chain of bapineuzumab, not including signal sequence, IgG1 isotype, no Fc mutations

(SEQ ID NO: 22)
EVQLLESGGGLVQPGGSLRLSCAASGFTFSNYGMSWVRQAPGKGLEW
VASIRSGGGRTYYSDNVKGRFTISRDNSKNTLYLQMNSLRAEDTAVYY
CVRYDHYSGSSDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPGK

The C-terminal K residue can be absent, as indicated below.

(SEQ ID NO: 23)
EVQLLESGGGLVQPGGSLRLSCAASGFTFSNYGMSWVRQAPGKGLEW
VASIRSGGGRTYYSDNVKGRFTISRDNSKNTLYLQMNSLRAEDTAVYY
CVRYDHYSGSSDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPG

In some antibodies, positions 234, 235, and 237 of a human IgG heavy chain constant region can be AAA respectively, LLA respectively, LAG respectively, ALG respectively, AAG respectively, ALA respectively, or LAA respectively. As shown above, AAB-003 is an L234A, L235A, and G237A variant of bapineuzumab (i.e., having identical amino acid sequences to bapineuzumab except for the L234A, L235A, and G237A mutations, alanine (A) being the variant amino acid). Like bapineuzumab, AAB-003 has a full-length human kappa light chain constant region and a full-length human IgG1 heavy chain constant region (in either bapineuzumab or AAB-003, a C-terminal lysine residue is sometimes cleaved intracellularly and is sometimes missing from the final product).

Amino acids in the constant region are numbered by alignment with the human antibody EU (see Cunningham et al., J. Biol. Chem., 9, 3161 (1970)). That is, the heavy and light chains of an antibody are aligned with the heavy and light chains of EU to maximize amino acid sequence identity and each amino acid in the antibody is assigned the same number as the corresponding amino acid in EU. The EU numbering system is conventional (see generally, Kabat et al., Sequences of Protein of Immunological Interest, NIH Publication No. 91-3242, US Department of Health and Human Services (1991)).

The affinity of an antibody for complement component C1q can be altered by mutating at least one of the amino acid residues 318, 320, and 322 of the heavy chain to a residue having a different side chain. Other suitable alterations for altering, e.g., reducing or abolishing, specific C1q-binding to an antibody include changing any one of residues 318 (Glu), 320 (Lys) and 322 (Lys), to Ala. C1q binding activity can be abolished by replacing any one of the three specified residues with a residue having an inappropriate functionality on its side chain. It is not necessary to replace the ionic residues only with Ala to abolish C1q binding. It is also possible to use other alkyl-substituted non-ionic residues, such as Gly, Ile, Leu, or Val, or such aromatic non-polar residues as Phe, Tyr, Trp and Pro in place of any one of the three residues in order to abolish C1q binding. In addition, it is also be possible to use such polar non-ionic residues as Ser, Thr, Cys, and Met in place of residues 320 and 322, but not 318, to abolish C1q binding activity. Replacement of the 318 (Glu) residue by a polar residue may modify but not abolish C1q binding activity. Replacing residue 297 (Asn) with Ala results in removal of lytic activity while only slightly reducing (about three fold weaker) affinity for C1q. This alteration destroys the glycosylation site and the presence of carbohydrate that is required for complement activation. Any other substitution at this site also destroys the glycosylation site.

Additional mutations that can affect C1q binding to the constant region of human IgG1 include those described, e.g., in WO 06/036291. In this case, at least one of the following substitutions can be made to reduce C1q binding: D270A, K322A, P329A, and P311S. Each of these mutations, including those at residues 297, 318, and 320 can be made individually or in combination.

Antibodies with heavy chain constant region mutations that reduce binding to Fcγ receptor(s) and/or C1q can be used in any of the methods of the invention. Preferably, such antibodies have reduced binding relative to an otherwise identical antibody lacking the mutation of at least 50% to at least one Fcγ receptor and/or to C1q.

### B. Active Immunotherapy

Numerous fragments of Aβ have been now been described in the scientific and patent literature as agents for active immunotherapy (see, e.g., U.S. Pat. No. 6,750,324, US 20040213800; US 20070134762). In general, fragments including an epitope within residues 1-11 of Aβ induce antibodies that bind Fcγ receptors and induce a clearing response against amyloid deposits, whereas fragments lacking an epitope within residues 1-11 of Aβ induce antibodies that bind preferentially or exclusively to soluble forms of Aβ rather than plaques and induces little if any clearing response against amyloid deposits.

Preferred fragment for inducing antibodies that bind to amyloid deposits and induce a clearing response are N-terminal fragments beginning at residues 1-3 of Aβ and ending at residues 7-11 of Aβ. Exemplary N-terminal fragments include Aβ1-5, 1-6, 1-7, 1-10, 3-7, 1-3, and 1-4 with 1-7 being particularly preferred. A class of exemplary fragments includes fragments beginning at a residue between 1-3 (inclusive) and ending at a residue between 7-11 (inclusive).

Preferred fragments for inducing antibodies to soluble Aβ, which induce little, if any, clearing response against amyloid deposits include Aβ15-21, Aβ16-22, Aβ17-23, Aβ18-24, Aβ19-25, Aβ15-22, Aβ16-23, Aβ17-24, Aβ18-25, Aβ15-23, Aβ16-24, Aβ17-25, Aβ18-26, Aβ15-24, Aβ16-25, and A315-25. Aβ16-23 is particularly preferred meaning s a fragment including residues 16-23 of Aβ and lacking other residues of Aβ. Also preferred are C-terminal fragments of Aβ42 or 43 of 5-10 and preferably 7-10 contiguous amino acids. Analogous C-terminal fragments of Aβ40, or 39 can also be used. These fragments can generate an antibody response that includes end-specific antibodies. Fragments preferably lack T-cell epitopes that would induce T-cells against Aβ. Generally, T-cell epitopes are greater than 10 contiguous amino acids. Therefore, preferred fragments of Aβ are of size 5-10 or preferably 7-10 contiguous amino acids; i.e., sufficient length to generate an antibody response without generating a T-cell response. Absence of T-cell epitopes is preferred because these epitopes are not needed for immunogenic activity of fragments, and may cause an undesired inflammatory response in a subset of patients.

Agents to induce antibodies to Aβ that can be used in the methods of the invention also include (i) ACI-24 (AC Immune); (ii) Affitopes AD02 and AD02 (Affiris GmbH); (iii) Arctic Immunotherapeutic KLVFFAGDV (SEQ ID NO: 92) (BioArctic Neuroscience/Eisai); (iv) Aβ1-15-K-K-Aβ1-15 (Brigham & Women's Hospital); (v) 3-Vax™ and Recall-Vax™ (Intellect Neurosciences); (vi) K6-Aβ1-30 (Intellect Neurosciences/NYU); (vii) V-950 (Merck); (viii) CAD 106 (Novartis/Cytos); (ix) Aβ DCtag™ nanoparticle adjuvant (Prana Biotechnology/PRIMABioMed); (x) PX106 (also 2Aβ1-11-PADRE, from Pharmexa/Lundbeck); (xi) A34-10 conjugated to a T cell epitope (U. Toronto); and (xii) p3102 and p3075 (United Biomedical).

ACI-24 is an Aβ1-15 liposome construct with Aβ1-15-K-K-16C palmitic acid inserted into a liposomal bilayer. These compounds are described in US 2004/0242845, WO 05/081872, US 2007/0281006, and US 2006/0073158. Affitopes AD01 and AD02 are mimotopes from the N-terminus of Aβ, as described in WO 06/005707. The Arctic Immunotherapeutic is derived from Aβ22 of E692G, as described in US 20020162129 and US 20070248606. Aβ1-15-K-K-Aβ1-15 represents two linked N-terminal Aβ fragments, as described in WO 05/012330 and WO 02/0123553. β-Vax™, Recall-Vax™ and K6-Aβ1-30 are Aβ fragments linked to a T cell epitope, as described in WO 01/42306. V-950 is an 8-mer Aβ peptide linked to a multivalent linear peptide with at least one spacer and a multivalent branched multiple antigen peptide, as described in WO 06/121656. CAD106 is a Qβ carrier (an RNA VLP) linked to an N-terminal Aβ peptide, as described in WO 04/016282. The Aβ DCtag™ nanoparticle adjuvant is described, e.g., in WO 02/00245. PX106 is a Aβ1-11 peptide linked to a T cell epitope called a “pan DR epitope peptide (PADRE),” as described in U.S. Pat. No. 7,135,181. p3102 and p3075 are Aβ1-14 peptides linked by a spacer to a T cell epitope (e.g., measles epitope), as described in US 20030068325 US 20040247612, U.S. Pat. No. 6,906,169, and WO 02/096350.

Fragments are usually fragments of natural Aβ (SEQ ID NO:1) but can include unnatural amino acids or modifications of N or C terminal amino acids at a one, two, five, ten or even all positions. For example, the natural aspartic acid residue at position 1 and/or 7 of Aβ can be replaced with iso-aspartic acid. Examples of unnatural amino acids are D, alpha, alpha-disubstituted amino acids, N-alkyl amino acids, lactic acid, 4-hydroxyproline, γ-carboxyglutamate, epsilon-N,N,N-trimethyllysine, epsilon-N-acetyllysine, O-phosphoserine, N-acetylserine, N-formylmethionine, 3-methylhistidine, 5-hydroxylysine, omega-N-methylarginine, β-alanine, ornithine, norleucine, norvaline, hydroxproline, thyroxine, γ-amino butyric acid, homoserine, citrulline, and isoaspartic acid. Some therapeutic agents of the invention are all-D peptides, e.g., all-D Aβ or all-D Aβ fragment, and all-D peptide analogs. Fragments can be screened for prophylactic or therapeutic efficacy in transgenic animal models in comparison with untreated or placebo controls.

Fragments are typically conjugated to carrier molecules, which provide a T-cell epitope, and thus promote an immune response against the fragment conjugated to the carrier. A single agent can be linked to a single carrier, multiple copies of an agent can be linked to multiple copies of a carrier, which are in turn linked to each other, multiple copies of an agent can be linked to a single copy of a carrier, or a single copy of an agent can be linked to multiple copies of a carrier, or different carriers. Suitable carriers include serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking agents of the invention to an immunostimulatory polymer molecule (e.g., tripalmitoyl-5-glycerine cysteine (Pam₃Cys), mannan (a mannose polymer), or glucan (a β 1→2 polymer)), cytokines (e.g., IL-1, IL-1 alpha and β peptides, IL-2, γ-INF, IL-10, GM-CSF), and chemokines (e.g., MIP1-α and β, and RANTES) Immunogenic agents can also be linked to peptides that enhance transport across tissues, as described in O'Mahony, WO 97/17613 and WO 97/17614.

Immunogens may be linked to the carries with or with out spacers amino acids (e.g., gly-gly).

Additional carriers include virus-like particles. Virus-like particles (VLPs), also called pseudovirions or virus-derived particles, represent subunit structures composed of multiple copies of a viral capsid and/or envelope protein capable of self assembly into VLPs of defined spherical symmetry in vivo. (Powilleit, et al., (2007) PLoS ONE 2(5):e415.) These particles have been found to be useful as antigen delivery systems. VLPs can be produced and readily purified in large quantities and due to their particulate nature and high molecular weights. VLPs induce an immune response without additional application of an adjuvant. (Ulrich et al., (1996) Intervirology 39:126-132.) Exemplary chimeric particles useful as VLP antigen delivery systems include those based on hepatitis B virus, human immunodeficiency virus (HIV), yeast retrotransposon Ty, yeast totivirus L-A, parvovirus, influenza virus, Norwalk virus, rotavirus, adeno-associated virus, bluetongue virus, hepatitis A virus, human papillomavirus, measles virus, polyoma virus and RNA phage virus, as well as those based on various retroviruses and lentiviruses. For review, see Lechner, et al. (2002) Intervirology 45:212-217.

The core protein of hepatitis B virus (HBcAg) is a common VLP used for carrying foreign antigens (see Koletzki et al., (1997) J Gen Vir 78:2049-2053). Briefly, HBcAg can be used as a core to construct VLPs that present extended foreign protein segments. The method employs a construct having a linker sequence between the a C-terminally truncated HBcAg and a foreign protein sequence that contains a stop codon. Truncated HBcAg/foreign protein chimera is expressed utilizing a read through mechanism based on the opal TGA-Trp mutation for expression in an E. coli suppressor strain. The method described by Koletzki et al. allows for incorporation of long foreign protein sequences into VLPs, allowing for a greater variety of antigens to be carried by the VLP.

The HIV virus Gag protein can be used as an antigen carrier system (see Griffiths et al., (1993) J. Virol. 67(6):3191-3198). Griffiths utilized the V3 loop of HIV, which is the principle neutralizing determinant of the HIV envelope. The Gag:V3 fusion proteins assembled in vivo into hybrid Gag particles, designated virus-derived particles (VDPs). The VDPs induce both humoral and cellular responses. As the V3 loop contains a CTL epitope, immunization with Gag:V3 induces a CTL response to the V3 protein portion of the VLP.

A hybrid HIV:Ty VLP can also be used (see Adams et al., (1987) Nature 329(3):68-70). The HIV:Ty VLP employs the p1 protein of the yeast transposon Ty. The first 381 amino acids of p1 are sufficient for VLP formation. The HIV:Ty fusion proteins are capable of assembling into VLPs in vivo, as well as inducing an immune response to the HIV antigen carried by the VLP. VLPs using the Ty p1 protein can also contain p1 fused to the whole of an alpha2-interferon, the product of the bovine papilloma virus E1 and E2 genes, and a portion of an influenza hemagglutinin. Each of these Ty fusions formed VLPs and were capable of inducing production of antisera to the non-Ty VLP component.

VLPs can also be designed from variants of the yeast totivirus L-A (see Powilleit et al. (2007) PLOS One 2(5):e415). The Pol gene of the L-A virus can be replaced with an appropriate antigen to induce a specific immune response, demonstrating that yeast VLPs are effective antigen carriers.

Recombinant, nonreplicative parvovirus-like particles can also be used as antigen carriers. (Sedlik, et al. (1997) PNAS 94:7503-7508.) These particles allow the carried antigens into the cytosol so they enter the class I-restricted immunological pathway, thus stimulating cytotoxic T-lymphocyte (CTL) mediated responses. Sedlik specifically used PPV:VLP, which contained the VP2 capsid protein of the parvovirus and residues 118-132 from the lymphocytic choriomeningitis virus (LCMV) was inserted into the VP2 capsid protein. The PPV:VLP containing LCMV was capable of inducing an immune response to LCMV and elicited immunological protection against lethal viral doses in pre-immunized mice.

VLPs can also comprise replication incompetent influenza that lack the influenza NS2 gene, the gene essential for viral replication. (Watanabe, et al. (1996) J. Virol. 76(2):767-773.) These VLPs infect mammalian cells and allow expression of foreign proteins.

Norwalk virus (NV)-based VLPs can also be used as vehicles for immunogen delivery. (Ball, et al. (1999) Gastroenterology 117:40-48.) The NV genome has three open reading frames (ORFs 1-3). Recombinant baculovirus expression of ORFs 2 and 3 allows for spontaneous assembly of high yields of recombinant Norwalk virus (rNV) VLPs.

Some conjugates can be formed by linking agents of the invention to at least one T cell epitope. Some T cell epitopes are promiscuous whereas other T cell epitopes are universal. Promiscuous T cell epitopes are capable of enhancing the induction of T cell immunity in a wide variety of subjects displaying various HLA types. In contrast to promiscuous T cell epitopes, universal T cell epitopes are capable of enhancing the induction of T cell immunity in a large percentage, e.g., at least 75%, of subjects displaying various HLA molecules encoded by different HLA-DR alleles.

A large number of naturally occurring T-cell epitopes exist, such as, tetanus toxoid (e.g., the P2 and P30 epitopes), Hepatitis B surface antigen, pertussis, toxoid, measles virus F protein, Chlamydia trachomatis major outer membrane protein, diphtheria toxoid, Plasmodium falciparum circumsporozoite T, Plasmodium falciparum CS antigen, Schistosoma mansoni triose phosphate isomerase, Escherichia coli TraT, and Influenza virus hemagglutinin (HA). The immunogenic peptides of the invention can also be conjugated to the T-cell epitopes described in Sinigaglia F. et al., Nature, 336:778-780 (1988); Chicz R. M. et al., J. Exp. Med., 178:27-47 (1993); Hammer J. et al., Cell 74:197-203 (1993); Falk K. et al., Immunogenetics, 39:230-242 (1994); WO 98/23635; and, Southwood S. et al. J. Immunology, 160:3363-3373 (1998).

Carriers also include virus-like particles (see US 20040141984).

Fragments are often administered with pharmaceutically acceptable adjuvants. The adjuvant increases the titer of induced antibodies and/or the binding affinity of induced antibodies relative to the situation if the peptide were used alone. A variety of adjuvants can be used in combination with an immunogenic fragment of Aβ, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Mont., now part of Corixa). Stimulon™ QS-21 is a triterpene glycoside or saponin isolated from the bark of the Quillaj a Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, Mass.; now Antigenics, Inc., New York, N.Y.). Other adjuvants are oil in water emulsions (such as squalene or peanut oil), optionally in combination with immune stimulants, such as monophosphoryl lipid A (see Stoute et al., N Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

A preferred class of adjuvants is aluminum salts (alum), such as alum hydroxide, alum phosphate, alum sulfate. Such adjuvants can be used with or without other specific immunostimulating agents such as MPL or 3-DMP, QS-21, polymeric or monomeric amino acids such as polyglutamic acid or polylysine. Another class of adjuvants is oil-in-water emulsion formulations. Such adjuvants can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn-glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) theramide™), or other bacterial cell wall components. Oil-in-water emulsions include (a) MF59 (WO 90/14837), containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.), (b) SAF, containing 10% Squalene, 0.4% Tween 80, 5% pluronic-blocked polymer L121, and thr-MDP, either microfluidized into a submicron emulsion or vortexed to generate a larger particle size emulsion, and (c) Ribi™ adjuvant system (RAS), (Ribi ImmunoChem, Hamilton, Mont.) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™).

Another class of preferred adjuvants is saponin adjuvants, such as Stimulon™ (QS-21, Aquila, Framingham, Mass.) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

An adjuvant can be administered with an immunogen as a single composition, or can be administered before, concurrent with or after administration of the immunogen. Immunogen and adjuvant can be packaged and supplied in the same vial or can be packaged in separate vials and mixed before use. Immunogen and adjuvant are typically packaged with a label indicating the intended therapeutic application. If immunogen and adjuvant are packaged separately, the packaging typically includes instructions for mixing before use. The choice of an adjuvant and/or carrier depends on the stability of the immunogenic formulation containing the adjuvant, the route of administration, the dosing schedule, the efficacy of the adjuvant for the species being vaccinated, and, in humans, a pharmaceutically acceptable adjuvant is one that has been approved or is approvable for human administration by pertinent regulatory bodies. For example, Complete Freund's adjuvant is not suitable for human administration. Alum, MPL and QS-21 are preferred. Optionally, two or more different adjuvants can be used simultaneously. Preferred combinations include alum with MPL, alum with QS-21, MPL with QS-21, MPL or RC-529 with GM-CSF, and alum, QS-21 and MPL together. Also, Incomplete Freund's adjuvant can be used (Chang et al., Advanced Drug Delivery Reviews 32, 173-186 (1998)), optionally in combination with any of alum, QS-21, and MPL and all combinations thereof.

### C. Immunotherapy Regimes

In prophylactic applications, agents or pharmaceutical compositions or medicaments containing the same are administered to a patient susceptible to, or otherwise at risk of, Alzheimer's disease in regime (dose, frequency and route of administration) effective to reduce the risk, lessen the severity, or delay the outset of at least one sign or symptom of the disease. In particular, the regime is preferably effective to reduce the amount of amyloid deposits or at least inhibit increase of the amount of amyloid deposits in the brain of the patient. Patients at risk of Alzheimer's disease include patients with above normal levels of amyloid deposits in the brain who have not been diagnosed with Alzheimer's disease and patients with mild cognitive impairment who have not been diagnosed with Alzheimer's disease. In therapeutic applications, agent compositions or medicaments are administered to a patient suspected of, or already suffering from Alzheimer's disease in a regime (dose, frequency and route of administration) effective to ameliorate or at least inhibit further deterioration of at least one sign or symptom of the disease. In particular, the regime is preferably effective to reduce or at least inhibit further increase of amyloid deposits in the patients.

Effective doses of vary depending upon many different factors, including means of administration, target site, physiological state of the patient, whether the patient is human or an animal, other medications administered, and whether treatment is prophylactic or therapeutic.

An exemplary dosage range for antibodies is from about 0.01 to 5 mg/kg, and more usually 0.1 to 3 mg/kg or 0.15-2 mg/kg or 0.15-1.5 mg/kg, of patient body weight. Antibody can be administered such doses daily, on alternative days, weekly, biweekly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

For intravenous administration, doses of 0.1 mg/kg to 2 mg/kg, and preferably 0.5 mg/kg or 1.5 mg/kg administered intravenously quarterly are suitable. Preferred doses of antibody for monthly intravenous administration occur in the range of 0.1-1.0 mg/kg antibody or preferably 0.5-1.0 mg/kg antibody.

For more frequent dosing, e.g., from weekly to monthly dosing, subcutaneous administration is preferred. Subcutaneous dosing is easier to administer and can reduce maximum serum concentrations relative to intravenous dosing. The doses used for subcutaneous dosing are usually in the range of 0.01 to 0.6 mg/kg or 0.01-0.35 mg/kg, preferably, 0.05-0.25 mg/kg. For weekly or biweekly dosing, the dose is preferably in the range of 0.015-0.2 mg/kg, or 0.05-0.15 mg/kg. For weekly dosing, the dose is preferably 0.05 to 0.07 mg/kg, e.g., about 0.06 mg/kg. For biweekly dosing, the dose is preferably 0.1 to 0.15 mg/kg. For monthly dosing, the dose is preferably 0.1 to 0.3 mg/kg or about 0.2 mg/kg. Monthly dosing includes dosing by the calendar month or lunar month (i.e., every four weeks). Here as elsewhere in the application, dosages expressed in mg/kg can be converted to absolute mass dosages by multiplying by the mass of a typical patient (e.g., 75 kg) typically rounding to a whole number. Other regimes are described by e.g., PCT/US2007/009499. The dosage and frequency can be varied within these guidelines based on the ApoE status of the patient as discussed above.

The amount of an agent for active administration varies from 1-500 μg per patient and more usually from 5-100 μg per injection for human administration. Exemplary dosages per injection are 3, 10, 30, or 90 μg for each human injection. The mass of immunogen also depends on the mass ratio of immunogenic epitope within the immunogen to the mass of immunogen as a whole. Typically, 10⁻³ to 10⁻⁵ micromoles of immunogenic epitope are used for each immunization of immunogen. The timing of injections can vary significantly from once a day, to once a year, to once a decade. On any given day that a dosage of immunogen is given, the dosage is greater than 1 μg/patient and usually greater than 10 μg/patient if adjuvant is also administered, and greater than 10 μg/patient and usually greater than 100 μg/patient in the absence of adjuvant. A typical regimen consists of an immunization followed by booster injections at time intervals, such as 6 week intervals. Another regimen consists of an immunization followed by booster injections 1, 2 and 12 months later. Another regimen entails an injection every two months for life Alternatively, booster injections can be on an irregular basis as indicated by monitoring of immune response. The dosage and frequency can be varied such that antibodies induced by an active agent have mean serum concentrations within a range of 0.1-60, 0.4-20, or 1-15 or 2-7 μg/ml as in passive administration. The dosage and frequency can be varied within these guidelines based on the ApoE status of the patient as discussed above.

### EXAMPLES

This example shows bapineuzumab-related changes in cortical Aβ deposits in vivo using [¹¹C]PiB PET imaging.

### Methods

### Study Design

The clinical trial was a phase 2, multicenter, randomized, double-blind, placebo-controlled, multiple-ascending dose study. Patients were randomly assigned to receive either intravenous (IV) bapineuzumab or placebo, in one of three dose cohorts (0.5 [A], 1.0 [B], or 2.0 [C] mg/kg). Up to 30 patients were planned for enrollment (10 per dose cohort with patients in each dose cohort [A, B, or C] receiving bapineuzumab or placebo in a 7:3 ratio). Patients who completed the screening phase and met all inclusion criteria were eligible for randomization. 28 patients were enrolled in the study (10 in cohort A, 10 in cohort B and eight in cohort C). The sponsor terminated enrollment in cohort C following the observation of more frequent cerebral vasogenic edema at the 2.0 mg/kg dose in other studies. Randomized patients received study drug as a 1-hour IV infusion every 13 weeks for up to six infusions. Each patient underwent [¹¹C]PiB PET, [¹⁸F]FDG PET, clinical assessments of cognition and function, cerebrospinal fluid (CSF) sampling for Aβ and tau, volumetric and safety magnetic resonance imaging (MRI), and safety evaluations. The final assessment was at week 78.

### Patients

Eligible patients were aged 50 to 80 years inclusive and met NINCDS-ADRDA (National Institute of Neurological and Communicative Disorders and Stroke—Alzheimer's Disease and Related Disorders Association [now known as the Alzheimer's Association]) criteria for probable AD. (McKhann, Neurology; 34:939-944 (1984). In addition, patients were required to have Aβ burden at baseline in the typical range expected for AD patients, defined as [¹¹C]PiB PET retention ratios relative to cerebellum ≧1.5 in at least three brain regions among the anterior cingulate, posterior cingulate, frontal, temporal, and parietal cortices. Additional inclusion criteria were an MRI consistent with AD, a Mini-Mental State Exam (MMSE) score of 18-26, (Folstein, J Psychiatr Res; 12:189-198 (1975) and a Rosen Modified Hachinski Ischemic score ≦4. (Rosen, Ann Neurol; 7:486-488 (1980). Patients were excluded for clinically significant neurological disease other than AD; a major psychiatric disorder, history of stroke or seizures, a Hamilton Rating Scale score for Depression >12; (Hamilton, J Neurol Neurosurg Psychiatry; 23:56-62 (1960) current anticonvulsant, antiparkinsonian, anticoagulant, or narcotic medications; recent immunosuppressive or cancer chemotherapy medications; or cognitive enhancers other than acetylcholinesterase inhibitors or memantine at a stable dose for at least 120 days before screening.

### [¹¹C]PiB PET Methods

Details of the synthesis of [¹¹C]PiB and acquisition of PiB PET data have been previously described. Edison et al. Neurology; 68:501-508 (2007). Briefly, all [¹¹C]PiB images were acquired using a Siemens ECAT EXACT HR+ scanner after an attenuation scan that preceded an IV bolus of approximately 370 MBq [¹¹C]PiB (specific activity ≧10 GBq/μmol at injection). The images were acquired in 32 frames over 90 minutes. Cortex:cerebellar ratio images of [¹¹C]PiB retention were generated at a single site (Hammersmith Imanet Ltd, GE Healthcare) using data from 60-90 minutes post-injection as previously reported. Edison et al., supra. [¹¹C]PiB PET images were co-registered to the individual's MRI, which was normalized into standard Montreal Neurological Institute (MNI) space. A probabilistic brain atlas was used to create a standard template of regions of interest (ROIs) for sampling segmented grey matter regions. (Hammers, et al. Hum Brain Mapp; 19:224-247 (2003). For analysis, six predefined cortical ROIs were included: the anterior cingulate, posterior cingulate, frontal, temporal, parietal, and occipital cortices. The average of all six ROIs was also calculated ([¹¹ C]PiB average). [¹¹C]PiB PET scans were obtained at screening and weeks 20, 45, and 78.

### [¹⁸F]FDG PET, Clinical, CSF and MRI Outcome Measures

Parametric images of regional cerebral glucose metabolism (rCMR_{glc}) relative to brainstem were generated from the brain [¹⁸F]FDG time activity curves between 35-55 minutes after tracer injection. The parametric rCMR_{glc} images were transformed into MNI stereotaxic space, and a probabilistic atlas was used to define six cortical ROIs and their average ([¹⁸F]FDG average), as for the [¹¹C]PiB analysis, at screening and at week 78.

The Alzheimer's Disease Assessment Scale-Cognitive subscale (ADAS-Cog), (Rosen, Am J Psychiatry; 141:1356-1364 (1984); Mohs., Alzheimer Dis Assoc Disord; 11(Suppl 2):S13-S21 (1997). Disability Assessment for Dementia (DAD), (Gauthier, Int Psychogeriatr; 9(Suppl 1):163-165 (1997). Neuropsychological Test Battery (NTB), (Harrison, Arch Neurol; 64:1323-1329 (2007) and MMSE (range 0-30) scales were administered approximately every 3 months; the Clinical Dementia Rating-Sum of Boxes (CDR-SB; range 0-18) (Morris J C., Neurology; 43:2412-2414 (1993) and Neuropsychiatric Inventory (NPI) (Cummings, Neurology; 44:2308-2314 (1994) were administered every 6 months. In patients consenting to lumbar puncture, CSF was obtained before treatment and at week 52. CSF biomarkers were measured by sandwich ELISAs for total tau, (Blennow, Mol Chem Neuropathol; 26:231-245 (1995) phospho-tau (P-tau181), (Vanmechelen, Neurosci Lett; 285:49-52 (2000) and Aβ₄₂ (Andreasen, Arch Neurol; 56:673-680 (1999) (with the 4G8 antibody replacing 3D6 to measure Aβ_{X-42}). Volumetric and safety MRI scans were performed before treatment, at week 6, and then at 13-week intervals through week 71. Exploratory MRI outcomes included change in whole brain (BBSI) and ventricular volumes (VBSI) from baseline to week 71 as measured by the boundary shift integral (BSI) method. (Fox, Arch Neurol; 57:339-344 (2000).

### Statistical Analysis

### Primary Analysis

The prespecified primary analysis compared the pooled bapineuzumab and pooled placebo groups at week 78 using a repeated measures model (mixed model for repeated measures, MMRM). The response variable was the change from screening to weeks 20, 45, and 78 in the average [¹¹C]PiB cortical:cerebellar retention ratio across the six predefined cortical ROIs. The explanatory variables included treatment group, screening [¹¹C]PiB PET value as a continuous covariate, baseline MMSE category (high [22-26] vs low [18-21]), visit week (a categorical factor), and the interaction between treatment and visit week. The covariance matrix was chosen from a prespecified set based on Akaike's information criterion. The primary analysis was a two-sided test of the week 78 least squares mean difference with significance level α=0.05. The analysis included all patients in the modified intent-to-treat (MITT) analysis population, predefined as all randomized patients who received any amount of study drug and who had a screening and at least one valid post-baseline PET scan.

### Exploratory Analyses

The six individual [¹¹C]PiB PET ROIs were analyzed using the same method as the overall [¹¹C]PiB PET average. The change from screening in the [¹⁸F]FDG PET average was analyzed using analysis of covariance (ANCOVA) with model terms for treatment (pooled bapineuzumab vs pooled placebo), screening value, and baseline MMSE category. MRI and clinical endpoints were analyzed using the same method as [¹¹C]PiB PET average, except that the models for BBSI and VBSI included baseline whole brain volume and baseline ventricular volume as covariates, respectively. CSF variables were analyzed using the same ANCOVA approach as [¹⁸F]FDG PET.

Due to apparent differences between the treated and placebo groups on some baseline assessments (e.g., NTB, CDR-SB, and [¹¹C]PiB PET) additional analyses adjusted for these imbalances: the MMRM and ANCOVA analyses described above were repeated without the screening/baseline covariate but with the addition of model terms for baseline NTB, CDR-SB, and [¹¹C]PiB average and, in the MMRMs, the corresponding covariate-by-visit interactions. Exploratory analyses were not adjusted for multiple comparisons.

### Sample Size

Based on previously reported standardized uptake values, (Klunk. W E, Engler H, Nordberg A, Wang Y, Blomqvist G, Holt D P, et al., Ann Neurol; 55(3):306-319 (2004)), it was estimated that there would be greater than 97% power to detect a treatment difference of 0.25 in [¹¹C]PiB retention between pooled bapineuzumab and pooled placebo in the change from screening to week 78, using a two-sided t-test at the 5% significance level. The study was not powered to evaluate efficacy on clinical or other biomarker outcomes.

### Results

### Patient Disposition

Of 53 screened patients, 28 were randomized (20 bapineuzumab vs eight placebo; 10 in the 0.5 mg/kg cohort, 10 in the 1.0 mg/kg cohort, and eight in the 2.0 mg/kg cohort). Eight screening failures did not meet the inclusion criteria because of low [¹¹C]PiB retention. Fifteen patients failed to meet other inclusion/exclusion criteria, and two did not complete enrollment. All randomized patients received at least one dose of bapineuzumab or placebo (safety population). Among those dosed, 26 (19 bapineuzumab; seven placebo) had a baseline and at least one post-baseline [¹¹C]PiB assessment and were included in the MITT population. Eighteen bapineuzumab patients (90.0%) and six placebo (75.0%) patients were evaluated at week 78. Fifteen bapineuzumab (75.0%) and five (62.5%) placebo patients had [¹¹C]PiB assessments at week 78.

### Baseline Demographics and Assessments

Baseline characteristics are summarized for the MITT population by treatment group in Table 1. Baseline demographics were balanced between treatment groups. The baseline [¹¹C]PiB average of all six target regions trended lower for the pooled placebo group compared with the pooled bapineuzumab group (p=0.058). The same pattern held true for some individual ROIs, notably the anterior cingulate (p=0.029), frontal (p=0.040), posterior cingulate (p=0.077), and parietal cortex (p=0.053). Apparent baseline imbalances between the treatment groups were also observed on some of the clinical assessments, with evidence of milder disease (better performance) in the placebo group on the CDR-SB (p=0.007) and the NTB (p=0.040). Seventy-one percent (71.4%) of placebo-treated patients fell into the high MMSE category (22-26) compared with 36.8% of bapineuzumab-treated patients.

TABLE 1
Patient demographics and baseline characteristics (MITT population)

| | All bapineuzumab | | All placebo | | |
|---|---|---|---|---|---|
| | (N = 19) | | (N = 7) | | p value |
| Demographics/baseline | | | | | |
| characteristics | | | | | |
| Age, years (mean [SD]) | 67·3 | (8·60) | 70·0 | (8·81) | 0·481 |
| Gender, n (%) female | 8 | (42·1) | 4 | (57·1) | 0·665 |
| Race, n (%) white | 19 | (100·0) | 7 | (100·0) | 1·000 |
| Duration of AD, years (mean [SD]) | 3·4 | (2·04) | 3·4 | (2·45) | 0·971 |
| MMSE high (22-26), n (%) | 7 | (36·8) | 5 | (71·4) | 0·190 |
| ApoE4 status, n (%) carrier | 12 | (63·2) | 5 | (71·4) | 1·000 |
| AChEI or memantine use, n (%) | 19 | (100·0) | 7 | (100·0) | 1·000 |
| Imaging biomarkers, mean (SD) | | | | | |
| [¹¹C]PiB PET average | 2·06 | (0·200) | 1·89 | (0·193) | 0·058 |
| Anterior cingulate | 2·38 | (0·266) | 2·12 | (0·211) | 0·029* |
| Posterior cingulate | 2·37 | (0·241) | 2·16 | (0·302) | 0·077 |
| Frontal cortex | 2·10 | (0·225) | 1·88 | (0·207) | 0·040* |
| Temporal cortex | 1·83 | (0·209) | 1·72 | (0·217) | 0·255 |
| Parietal cortex | 2·03 | (0·229) | 1·83 | (0·206) | 0·053 |
| Occipital cortex | 1·66 | (0·269) | 1·60 | (0·202) | 0·620 |
| Whole brain volume (cc) | 1054·26 | (104·162) | 1051·67 | (149·453) | 0·963 |
| Ventricular volume (cc) | 57·17 | (21·648) | 45·98 | (41·357) | 0·550 |
| [¹⁸F]FDG PET average | 1·24 | (0·105) | 1·22 | (0·080) | 0·645 |
| Clinical efficacy measures, | | | | | |
| mean (SD) | | | | | |
| ADAS-cog 11-item | 22·26 | (7·649) | 19·19 | (5·273) | 0·339 |
| ADAS-cog 12-item | 31·26 | (7·075) | 27·33 | (6·667) | 0·215 |
| CDR-SB | 5·61 | (1·638) | 3·50 | (1·500) | 0·007^{†} |
| DAD | 84·38 | (11·953) | 93·78 | (8·239) | 0·069 |
| MMSE | 21·0 | (2·33) | 22·3 | (2·69) | 0·243 |
| NTB | -0·149 | (0·5416) | 0·478 | (0·8321) | 0·040^{†} |
| NPI | 8·1 | (8·01) | 5·3 | (4·27) | 0·388 |

*p < 0·05; baseline imbalances indicate less [¹¹C]PiB uptake in placebo group.
^{†}p < 0·05; imbalances indicate better performance in placebo group.
For continuous variables (represented as mean and SD), p values are calculated based on a two-sample t-test. For categorical variables (represented as counts and percentages), p values are calculated using Fisher's exact test.
AChEI = acetylcholinesterase inhibitor; AD = Alzheimer's disease; ADAS-Cog = Alzheimer's Disease Assessment Scale-Cognitive subscale; ApoE4 = apolipoprotein E4; CDR-SB = Clinical Dementia Rating-Sum of Boxes; DAD = Disability Assessment for Dementia; [¹⁸F]FDG = 2-fluoro-2-deoxy-D-glucose; MITT = modified intent-to-treat; MMSE = Mini-Mental State Exam; NPI = Neuropsychiatric Inventory; NTB = Neuropsychological Test Battery; PET = positron emission tomography; PiB = Pittsburgh Compound B; [¹¹C]PiB average = [¹¹C]PiB average of all six cortical regions of interest; SD = standard deviation.

[¹¹C]PiB PET results

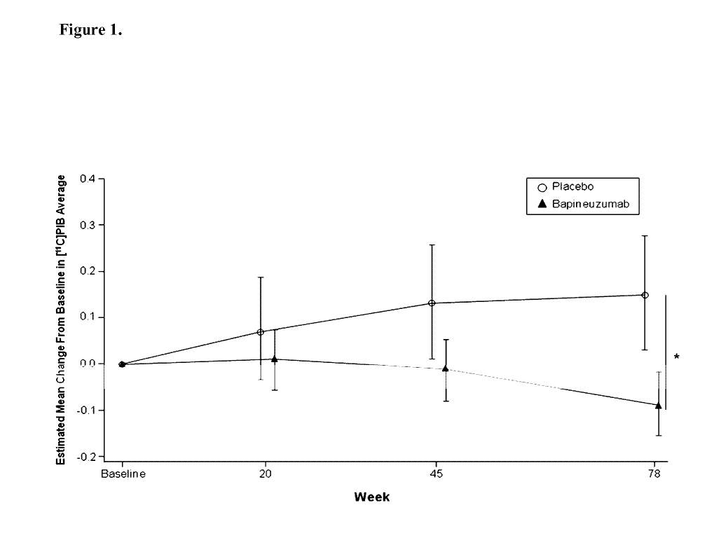

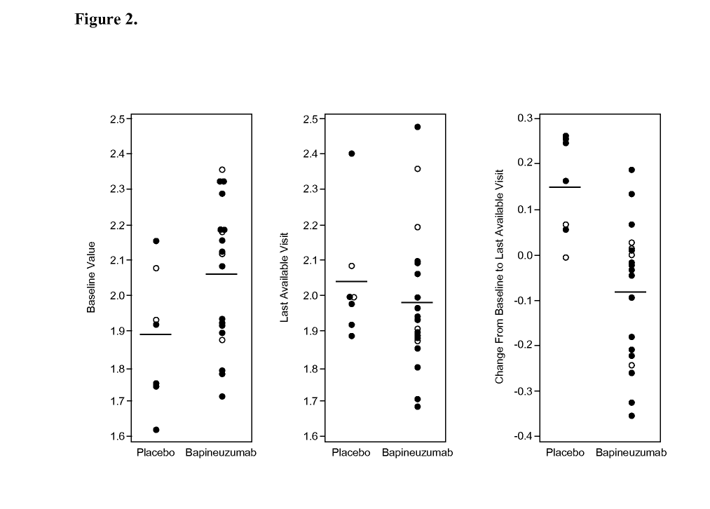

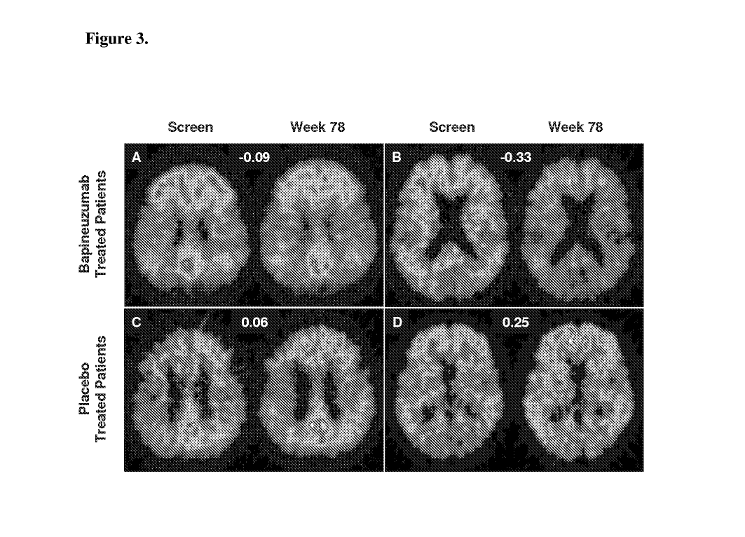

In the prespecified primary analysis (Table 2, first row), bapineuzumab-treated patients showed a significant reduction in [¹¹C]PiB average retention at week 78 compared with the placebo group (−0.24, p=0.003). A trend (p=0.059) was observed for the treatment-by-time interaction, suggesting that the treatment difference increased over time (FIG. 1). Within the bapineuzumab-treated group, a reduction in [¹¹C]PiB average retention at week 78 compared with baseline was observed (−0.09, 95% CI-0.157 to −0.019, p=0.014), while the placebo-treated group showed an increase (0.15, 95% CI 0.023 to 0.275, p=0.022). FIG. 2 shows [¹¹C]PiB PET retention for individual patients by treatment group at baseline and at their last available visit, as well as the change from baseline to last available visit. Individual patient [¹¹C]PiB scans before and after 78 weeks of treatment with bapineuzumab or placebo are shown in FIG. 3.

TABLE 2
PiB PET: [¹¹C]PiB average and PiB in individual regions of
interest - change from baseline to week 78 (MMRM, MITT population)

| | | | | | | Model | |
|---|---|---|---|---|---|---|---|
| | | | | Observed | | estimated | |
| | | | | treatment | Model | treatment | |
| Region of | | | Observed | difference | estimated | difference | |
| Interest | Treatment | N | mean (SD) | (95% CI) | mean | (95% CI) | p value |
| [¹¹C]PiB | Placebo | 7 | 0.20 | −0.29 | 0.15* | −0.24 | 0.003 |
| PET | | | (0.088) | (−0.450, −0.125) | | (−0.385, −0.089) | |
| average | Bapi | 1 | −0.09 | | −0.09* | | |
| | | 9 | (0.163) | | | | |
| Anterior | Placebo | 7 | 0.22 | −0.37 | 0.17 | −0.31 | 0.005 |
| cingulate | | | (0.133) | (−0.557, −0.179) | | (−0.523, −0.099) | |
| | Bapi | 1 | −0.15 | | −0.14^{†} | | |
| | | 9 | (0.184) | | | | |
| Posterior | Placebo | 7 | 0.24 | −0.33 | 0.16 | −0.25 | 0.014 |
| cingulate | | | (0.174) | (−0.530, −0.134) | | (−0.450, −0.054) | |
| | Bapi | 1 | −0.09 | | −0.09* | | |
| | | 9 | (0.185) | | | | |
| Frontal | Placebo | 7 | 0.23 | −0.31 | 0.16* | −0.24 | 0.006 |
| cortex | | | (0.078) | (−0.492, −0.128) | | (−0.409, −0.073) | |
| | Bapi | 1 | −0.08 | | −0.08 | | |
| | | 9 | (0.185) | | | | |
| Temporal | Placebo | 7 | 0.13 | −0.21 | 0.13* | −0.21 | 0.002 |
| cortex | | | (0.073) | (−0.359, −0.051) | | (−0.332, −0.083) | |
| | Bapi | 1 | −0.07 | | −0.08* | | |
| | | 9 | (0.156) | | | | |
| Parietal | Placebo | 7 | 0.23 | −0.32 | 0.15* | −0.23 | 0.004 |
| cortex | | | (0.064) | (−0.513, −0.120) | | (−0.384, −0.078) | |
| | Bapi | 1 | −0.09 | | −0.08* | | |
| | | 9 | (0.203) | | | | |
| Occipital | Placebo | 7 | 0.14 | −0.20 | 0.14^{†} | −0.20 | 0.001 |
| cortex | | | (0.059) | (−0.342, −0.049) | | (−0.315, −0.086) | |
| | Bapi | 1 | −0.06 | | −0.06* | | |
| | | 9 | (0.150) | | | | |

P value tests the model-estimated week 78 treatment difference using a two-sided test.
Model estimates are least squares means from the MMRM with change from baseline as the response and with model terms for treatment group with two levels (bapineuzumab and placebo), baseline score, baseline MMSE category, visit week (as a categorical variable), and the visit-by-treatment group interaction. The covariance matrix is chosen from a prespecified set based on Akaike's information criterion.
Negative within-group observed and estimated means indicate reduction of A□ compared with baseline. Negative observed and estimated treatment differences indicate reduced A□ from baseline in the bapineuzumab-treated group compared with placebo.
*p ≦ 0.05.
^{†}p ≦ 0.01 for the change from baseline within treatment group.
Bapi = bapineuzumab, CI = confidence interval, MITT = modified intent-to-treat, MMRM = mixed model for repeated measures, N = number of patients included in the model (MITT patients), PET = positron emission tomography; PiB = Pittsburgh Compound B; [¹¹C]PiB average = [¹¹C]PiB average of all six cortical regions of interest, SD = standard deviation.

The change in [¹¹C]PiB retention from baseline through week 78 for the six cortical ROIs showed results similar to those of the overall [¹¹C]PiB average (Table 2). Across the six ROIs, changes from baseline through week 78 showed consistent reductions in [¹¹C]PiB in the bapineuzumab-treated patients and increases in placebo-treated patients. The magnitude of treatment difference in the [¹¹C]PiB average was similar for each of the three doses: 0.5 mg/kg dose ^{˜}(0.24, p=0.009), 1.0 mg/kg dose ^{˜}(0.18, p=0.051), and 2.0 mg/kg dose ^{˜}(0.29, p=0.003).

The significant treatment difference observed in [¹¹C]PiB retention between the treated group and placebo group was maintained in the analyses adjusting for baseline NTB, CDR-SB, and [¹¹C]PiB average ([¹¹C]PiB average estimated treatment difference −0.25, p=0.025). The [¹¹C]PiB average change from baseline in bapineuzumab-treated patients was similar for ApoE4 carriers (mean ^{˜}0.08; n=9) and ApoE4 non-carriers (mean ^{˜}0.10; n=6).

The possibility of bapineuzumab displacing [¹¹C]PiB from its binding site was eliminated by showing that bapineuzumab does not compete for [³H]PiB binding to AD brain homogenates or synthetic Aβ fibrils at concentrations of bapineuzumab several orders of magnitude above those achieved in vivo. Because physiological concentrations of bapineuzumab seldom exceed 50 ng/ml in CSF, concentrations of 0.5 to 200,000 ng/ml were tested to fully determine whether bapineuzumab would compete with PiB for binding sites on Aβ. The study was performed using both synthetic Aβ1-40 fibrils and homogenates of AD brain frontal cortex having heavy plaque deposits. A concentration of 1 nM [³H]PiB was chosen to replicate in vivo concentrations of [¹¹C]PiB during human PET studies. A control for [³H]PiB binding to the filter sheet in the absence of Aβ fibrils or AD brain tissue was included as well. Bapineuzumab had no detectable effect on [³H]PiB binding to AD brain or Aβ1-40 fibrils at concentrations up to 20,000 ng/ml.

### Exploratory Clinical, [¹⁸F]FDG, MRI, and CSF Outcomes

Treatment differences varied across the exploratory endpoints (Table 3). After adjusting for baseline imbalances on the NTB, CDR-SB, and [¹¹C]PiB, treatment differences (p<0.05) were maintained for all PiB PET variables; however, no treatment differences were noted on the clinical, [¹⁸F]FDG PET, MRI, or CSF endpoints.

TABLE 3
Treatment differences on [¹¹C]PiB, clinical, and biomarker endpoints
in prespecified analysis and after adjusting for baseline clinical scores
(NTB, CDR-SB) and [¹¹C]PiB average (MITT population)

| | Prespecified analysis | | | Adjusted analysis | | |
|---|---|---|---|---|---|---|
| | Treatment difference | | | Treatment difference | | |
| | (95% CI) | | p value | (95% CI) | | p value |
| PiB endpoints | | | | | | |
| [¹¹C]PiB average | −0·24 | (−0·385, −0·089) | 0·003 | −0·25 | (−0·466, −0·034) | 0·025 |
| Anterior cingulate | −0·31 | (−0·523, −0·099) | 0·005 | −0·31 | (−0·611, −0·017) | 0·039 |
| Posterior cingulate | −0·25 | (−0·450, −0·054) | 0·014 | −0·29 | (−0·566, −0·016) | 0·039 |
| Frontal cortex | −0·24 | (−0·409, −0·073) | 0·006 | −0·25 | (−0·489, −0·002) | 0·048 |
| Temporal cortex | −0·21 | (−0·332, −0·083) | 0·002 | −0·20 | (−0·390, −0·007) | 0·043 |
| Parietal cortex | −0·23 | (−0·384, −0·078) | 0·004 | −0·25 | (−0·484, −0·016) | 0·037 |
| Occipital cortex | −0·20 | (−0·315, −0·086) | 0·001 | −0·20 | (−0·388, −0·021) | 0·030 |
| Clinical endpoints | | | | | | |
| ADAS-cog 11 | −8·41 | (−17·924, 1·113) | 0·081 | −3·49 | (−16·862, 9·887) | 0·594 |
| ADAS-cog 12 | −7·62 | (−14·963, −0·273) | 0·042 | −3·40 | (−13·614, 6·824) | 0·511 |
| DAD | −1·02 | (−19·425, 17·387) | 0·910 | 15·20 | (−1·777, 32·167) | 0·079 |
| CDR-SB | 0·39 | (−2·727, 3·502) | 0·799 | 2·42 | (−1·839, 6·681) | 0·251 |
| NTB | −0·12 | (−0·735, 0·486) | 0·676 | 0·396 | (−0·3235, 1·1145) | 0·266 |
| NPI | −0·52 | (−7·975, 6·941) | 0·889 | 5·99 | (−4·087, 16·071) | 0·235 |
| MMSE | −3·02 | (−7·414, 1·381) | 0·178 | −0·62 | (−6·511, 5·272) | 0·836 |
| Biomarker endpoints | | | | | | |
| [¹⁸F]FDG (average) | −0·01 | (−0·063, 0·049) | 0·796 | 0·00 | (−0·080, 0·080) | 0·992 |
| BBSI | 0·57 | (−10·319, 11·460) | 0·914 | −3·32 | (−14·465, 7·832) | 0·554 |
| VBSI | 4·87 | (−0·643, 10·393) | 0·080 | 3·65 | (−1·302, 8·602) | 0·146 |
| CSF Aβₓ₋₄₂ | 52·9 | (−316·79, 422·63) | 0·745 | 126·0 | (−781·83, 1033·73) | 0·720 |
| CSF tau | −93·5 | (−272·69, 85·74) | 0·257 | −165·1 | (−542·26, 212·12) | 0·291 |
| CSF p-tau | −5·0 | (−25·31, 15·36) | 0·581 | −19·2 | (−39·82, 1·35) | 0·060 |

For PiB and clinical endpoints, the prespecified analysis was based on the week 78 treatment difference estimated using least squares means from an MMRM with change from baseline (screening) as the response and with model terms for treatment group with two levels (bapineuzumab and placebo), baseline score, baseline MMSE category, visit week (as a categorical variable), and the visit-by-treatment group interaction. The covariance matrix was chosen from a prespecified set based on Akaike's information criterion. The adjusted analysis removed the baseline covariate and added model terms for baseline NTB, CDR-SB, and [¹¹C]PiB average and the corresponding covariate-by-visit interactions. The MMRM analysis incorporates all MITT patients (N = 19 for bapineuzumab, N = 7 for placebo). For PiB endpoints, negative treatment differences indicate less PiB retention for bapineuzumab; for clinical endpoints, positive treatment differences favor bapineuzumab (due to conventions adopted for calculating change from baseline to represent improvement).
For BBSI and VBSI, the prespecified model was the same, except that estimates are based on week 71 (the final MRI visit), and instead of a baseline covariate, the model for BBSI included whole brain volume and for VBSI included baseline ventricular volume. The adjusted analysis removed these covariates and added model terms for baseline NTB, CDR-SB, and [¹¹C]PiB average and the corresponding covariate-by-visit interactions. The MMRM analysis incorporates all MITT patients (N = 19 for bapineuzumab, N = 7 for placebo). A negative treatment difference for BBSI indicates less brain volume loss in the bapineuzumab group compared with placebo. A positive treatment difference for VBSI indicates a greater ventricular volume increase in the bapineuzumab group compared with placebo.
For [¹⁸F]FDG (average), the prespecified analysis was based on the week 78 treatment difference estimated using least squares means from an ANCOVA with change from baseline as the response and with model terms for treatment group with two levels (bapineuzumab and placebo), baseline score, and baseline MMSE category. The adjusted analysis removed the baseline covariate and added model terms for baseline NTB, CDR-SB, and [¹¹C]PiB average. The analysis is based on available week 78 [¹⁸F]FDG (average) data (n = 17 bapineuzumab, n = 5 placebo). A positive treatment difference indicates greater [¹⁸F]FDG retention compared with baseline for the bapineuzumab group compared with placebo.
For CSF variables, the prespecified analysis was based on the week 52 treatment difference estimated using least squares means from an ANCOVA with change from baseline as the response and with model terms for treatment group with two levels (bapineuzumab and placebo), baseline score, and baseline MMSE category. The adjusted analysis removed the baseline covariate and added model terms for baseline NTB, CDR-SB, and [¹¹C]PiB average. The analysis is based on available week 52 CSF data (n = 7 bapineuzumab, n = 4 placebo). Negative treatment differences for CSF tau and p-tau indicate greater reduction at week 52 relative to baseline in the bapineuzumab group compared with placebo. Positive treatment differences for CSF Aβₓ₋₄₂ indicate an increase relative to baseline in the bapineuzumab group compared with placebo.
ADAS-Cog = Alzheimer's Disease Assessment Scale-Cognitive subscale; ANCOVA = analysis of covariance; BBSI = brain boundary shift integral; CDR-SB = Clinical Dementia Rating-Sum of Boxes; CI = confidence interval, CSF = cerebrospinal fluid; DAD = Disability Assessment for Dementia; [¹⁸F]FDG = 2-fluoro-2-deoxy-D-glucose; MITT = modified intent-to-treat; MMRM = mixed model for repeated measures, MMSE = Mini-Mental State Exam; NPI = Neuropsychiatric Inventory; NTB = Neuropsychological Test Battery; PiB = Pittsburgh Compound B; [¹¹C]PiB average = [¹¹C]PiB average of all six cortical regions of interest; VBSI = ventricular boundary shift integral.

### Safety Results

VE was experienced by two bapineuzumab-treated patients, both ApoE4 carriers (ApoE genotypes were 4/4 and 3/4), in the highest dose cohort (2.0 mg/kg); both patients were asymptomatic, the events were discovered through MRI surveillance, and each patient had received one dose of bapineuzumab prior to onset. Both patients were permanently discontinued, and the outcomes were both considered resolved.

### DISCUSSION

The study showed a significant treatment difference for bapineuzumab-treated patients versus placebo-treated patients on change from baseline in the [¹¹C]PiB PET average retention from six targeted ROIs. A reduction in [¹¹C]PiB retention relative to baseline was noted for the bapineuzumab-treated group, whereas an increase was observed in the placebo group. The magnitude of the treatment difference in [¹¹C]PiB retention was similar for each of the three doses tested, and the treatment difference appeared to increase over time. The results for each of the six cortical ROIs paralleled those of the overall average, with consistent reductions in [¹¹C]PiB retention in bapineuzumab-treated patients and increases in placebo-treated patients.

Cortex:cerebellum ratios are unitless, and a ratio of 1.0 indicates no specific [¹¹C]PiB retention and should be accounted for in calculations of percent change. For example, a decrease from 2.0 to 1.8 ratio units would represent a 20 0% decrease in specific [¹¹C]PiB retention [i.e., (2.0−1.8)/(2.0−1.0)]. Accordingly, the 0.09 ratio unit decrease for bapineuzumab over 78 weeks represents an 8.5% decline from the baseline value of 2.06, while the 0.15 unit increase for placebo represents a 16.9% elevation over the baseline value of 1.89. Using this approach, one can estimate that bapineuzumab treatment was associated with an ˜25% reduction in cortical fibrillar Aβ over the course of 78 weeks compared with placebo. N-terminal antibodies bind Aβ oligomers (Shankaret al., Nat Med; 14:837-842 (2008)) as well as diffuse and compact plaques, (Bard, Nat Med; 6:916-919 (2008); Schenk et al., Nature; 400:173-177 (1999)) whereas PiB binds only fibrillar Aβ, and binds diffuse plaques less avidly than compact plaques. (Klunk et al., Ann Neurol; 55(3):306-319 (2004); Ikonomovic et al., Brain; 131:1630-1645 (2008)). [¹¹C]PiB may therefore underestimate the effect of bapinauzumab on total Aβ burden. Consistent with the increasing treatment difference in [¹¹C]PiB retention over time, greater differences may result from extended treatment. The bapineuzumab-treated group showed a significant reduction in [¹¹C]PiB retention from baseline independent of the change in the placebo group.

Because of the relatively small number of patients enrolled, there were apparent baseline imbalances between the two treatment groups on some measures. Disease severity appeared somewhat greater in the bapineuzumab group compared with the placebo group. After adjustment for baseline imbalances in baseline [¹¹C]PiB PET retention and clinical scores, the [¹¹C]PiB PET treatment difference persisted, whereas no statistically significant differences were noted on the clinical or other biomarker outcomes.

All patent filings, other publications, websites, accession numbers and the like cited above are incorporated by reference in their entirety for all purposes to the same extent as if each individual item were specifically and individually indicated to be so incorporated by reference. If different variants of an a sequence are associated with an accession number at different times, the version associated with the accession number at the filing date of this application is meant. Likewise, the version of a website in existence at the filing date of this application is meant. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

1. A method of monitoring treatment of a patient receiving Aβ-directed immunotherapy comprising administering to the patient a small-molecule positron-emission-tomography ligand (PET ligand) that binds to an amyloid deposit comprising Aβ and detecting the PET ligand in the brain using PET to provide an indication of a level of amyloid deposits of Aβ in the brain of the patient.

2. The method of claim 1, wherein the PET ligand binds to the Congo-Red binding site of Aβ.

3. The method of claim 1, wherein the PET ligand binds to the Thioflavin-T (Th-T) binding site of Aβ.

4. The method of claim 1, wherein the PET ligand binds to the 2-(1-{6-[(2-fluoroethyl-(methyl)amino]-2-naphthyl}ethylidene)malononitrile (FDDNP) binding site of Aβ.

5-10. (canceled)

11. The method of claim 1, wherein the PET ligand is selected from the group consisting of [¹⁸F]AV-14, [¹⁸F]AV-144, [¹¹C]AZD2995, [¹⁸F]-AZD4694 and [¹⁸F]-SMIBR-W372.

12-18. (canceled)

19. The method of claim 1, wherein the administering and detecting steps are before and after commencement of the Aβ-directed immunotherapy, and the level of amyloid deposits of Aβ is reduced after commencement of the therapy.

20. The method of claim 19, wherein no significant change in a biomarker selected from the group consisting of FFDG, BBSI, VBSI, CSF Aβ42, CSF tau and CSF p-tau is detectable when the reduced level of amyloid deposits of Aβ is detected.

21. The method of claim 19, wherein no significant increase in a measure of cognitive function is detectable when the reduced level of amyloid deposits of Aβ is detected.

25. (canceled)

26. The method of claim 1, wherein the regime of Aβ-directed immunotherapy is adjusted in response to the monitoring.

27. The method of claim 26, wherein the immunotherapy is adjusted without regard to measured values, if any, of biomarkers selected from the group consisting of FFDG, BBSI, VBSI, CSF Aβ42, CSF tau and CSFp-tau and measured values, if any, of cognitive function.

28. The method of claim 26, wherein no significant effect of the Aβ-directed immunotherapy on a biomarker selected from the group consisting of FFDG, BBSI, VBSI, CSF Aβ42, CSF tau and CSFp-tau is detectable when the regime is adjusted.

29-39. (canceled)

40. The method of claim 1, wherein the Aβ-directed immunotherapy is effected by administration of AAB-003 to the patient.

41. The method of claim 1, wherein the Aβ-directed immunotherapy is effected by administration of an Aβ fragment linked to a heterologous carrier as a conjugate to the patient.

42. The method of claim 41, wherein the Aβ fragment is Aβ1-7.

43. The method of claim 42, wherein the carrier is CRM197.

44-45. (canceled)

46. The method of claim 1, wherein the Aβ-directed immunotherapy is selected from the group consisting of the catalytic antibody ABP 102 (Abzyme, from Abiogen Pharma); ACI-01 Ab7 C2 (AC Immune Genentech); AZD-3102 (AstraZeneca/Dyax); IVIg (Gammagard S/D Immune Globulin Intravenous (Human), from Baxter Bioscience); BAN 2401 (BioArctic Neuroscience AB/Eisai Co. Ltd.; R1450 (Hoffman-La Roche/MorphoSys); LY2062430 (Eli Lilly); h3D6 (Eli Lilly); ACU-5A5 (a ADDL mAb from Merck/Acumen); α-amyloidspheroid (ASPD) antibody (Mitsubishi Pharma Corp.); the antibody derived from PBMCs of an AN1792 patient (Neurimmune Therapeutics AG); BC05 (Takeda); the CEN701-CEN706 antibodies (Centocor/Johnson & Johnson); and PF-04360365 (also called RN-1219 (h2286), from Pfizer/Rinat Neurosciences).

47-50. (canceled)

51. The method of claim 1, wherein the patient is an ApoE4 carrier.

52. The method of claim 1, wherein the patient is a non-ApoE4 carrier.

53. (canceled)

54. A method of performing a clinical trial, comprising
assigning a population of no more than 50 patients having or at elevated risk of a disease characterized by amyloid deposits comprising Aβ in the brain to treatment and placebo groups;
administering Aβ-directed therapy to the treatment group and a placebo to the placebo group;
comparing amyloid deposits in the treatment and placebo groups before and after administration of treatment or placebo by PET scanning of a small molecule PET ligand that binds amyloid deposits comprising Abeta;
wherein the amyloid deposits in the treatment group are significantly reduced relative to the amyloid deposits in the placebo group.

55. (canceled)

56. A method of prophylaxis against Alzheimer's disease, comprising:
determining a level of amyloid deposits in the brain of a patient who has no known cognitive impairment or has mild cognitive impairment but has not been diagnosed with Alzheimer's disease by PET scanning of a small molecule PET ligand that binds amyloid deposits comprising Aβ; and
administering Aβ-directed immunotherapy to the patient in response to determining that the level of amyloid deposits in the brain of the patient exceeds a normal level.

57-65. (canceled)
